(19) United States
(12) Patent Application Publication
(10) Pub. No.: US 2024/0000780 A1
(43) Pub. Date: Jan. 4, 2024

(54) PED5 INHIBITOR SEMI-SOLID COMPOSITIONS AND METHODS OF MAKING AND USING THEREOF

(71) Applicant: Seattle Gummy Company, Renton, WA (US)

(72) Inventors: Feng WAN, Sammamish, WA (US); William Brenden Carlson, Shoreline, WA (US)

(73) Assignee: Seattle Gummy Company, Renton, WA (US)

(21) Appl. No.: 18/039,270

(22) PCT Filed: Dec. 1, 2021
(86) PCT No.: PCT/US2021/061446

§ 371 Date: May 29, 2023

(51) Int. Cl. A61K 31/4985; A61K 47/26; A61K 47/42; A61K 9/68; A61K 47/36

(52) U.S. Cl. CPC A61K 31/4985; A61K 47/26; A61K 47/42; A61K 9/0058; A61K 47/36

(57) ABSTRACT

A chewable composition comprising an active pharmaceutical ingredient (API) composition, a surfactant composition, a binding composition, and a gelling composition in a sufficient amount to provide a cohesive gelled product. The API composition comprises a PED5 inhibitor; the surfactant composition and the API composition have a weight ration from about 0.1 to about 15; and the binding composition comprises a mono- or di-saccharide, a sugar alcohol, an oligosaccharide, or a combination thereof. In one embodiment, the API composition comprises tadalafil, sildenafil, vardenafil, avanafil, or a combination thereof.

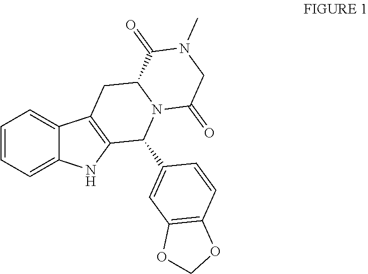 [SMILES: CN1CC(=O)N2[C@H](c3ccc4c(c3)OCO4)c3[nH]c4ccccc4c3C[C@@H]2C1=O]

### CROSS-REFERENCES TO RELATED APPLICATIONS

This application claims the benefit of priority from, and hereby incorporates by reference the entire disclosure, co-pending US Provisional Applications for (1) Patent Ser. No. 63/119,657, filed Dec. 1, 2020; (2) Patent Ser. No. 63/119,661, filed Dec. 1, 2020; (3) Patent Ser. No. 63/119,658, filed Dec. 1, 2020; and (4) and Patent Ser. No. 63/119,660, filed Dec. 1, 2020.

### TECHNICAL FIELD

This application relates to semi-solid edible or chewable gel compositions with one or more bioactive incorporated therein.

### BACKGROUND

Unless otherwise indicated herein, the materials described in this section are not prior art to the claims in this application and are not admitted being prior art by inclusion in this section.

Pharmaceuticals are available in a variety of dosage forms for treating the diseases. Dosages that are formulated to take orally including tablets, capsules, soft-gels, powders, chewable tablets, and liquid suspensions.

Tablets, capsules and soft-gels are difficult for individuals who have difficulties swallowing pills. This problem is magnified when the medications need to be taken 2-4 times per day to provide the desired therapeutic effect. Moreover, the need for a source of water or other liquid to assist with swallowing solid dosage forms can complicate administration.

Powders are often difficult to administer and chewable tablets can be hard to chew especially for seniors and young children. In addition, powders and chewable tablets often have an unpleasant after-taste.

Liquid suspensions or solutions are sometimes used as an alternative to solid oral dosage forms. However, the dosing with liquid dosage forms is not precise, which can lead to the administration of too little or too much medications. In addition, liquid dosage forms are messy and often have a bitter taste, which could impact person compliance.

Semi-solid chewable (gummy) composition could deliver medications and bioactive with an easier consumption profile. However, conventional gummy formulations are often packed with sugar and as a result having a high in glycemic index, making them unhealthy and potentially dangerous for diabetic patients.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid chewable composition. In one embodiment, the chewable composition includes an active pharmaceutical ingredient (API) composition comprising a PED5 inhibitor, a surfactant composition, wherein the surfactant composition and the API composition have a weight ration from about 0.1 to about 15, a binding composition, comprising a mono- or di-saccharide, a sugar alcohol, an oligosaccharide, or a combination thereof, and a gelling composition in a sufficient amount to provide a cohesive gelled product.

In one embodiment, the API composition comprises tadalafil, sildenafil, vardenafil, avanafil, or a combination thereof. In one embodiment, the API composition comprises DHEA. In one embodiment, the API composition comprises tadalafil.

In one embodiment, the API composition comprises an herbal ingredient with PED5 inhibiting activity such as epimedium (horny goat weed), its extract, powder or a combination thereof. In one embodiment, epimedium extract or powder is from about 2% to about 20% of the chewable composition.

In one embodiment, the chewable composition has from about 0.1% to 20%, 0.15% to 1%, 0.16% to 0.2% to about 0.5%, 1% to about 5%, 0.15% to about 3%, 2% to about 8%, or 5% to about 15% w/w of the API composition. In one embodiment, the chewable composition has about 2%, 0.16% to w/w of the API composition. In one embodiment, the chewable composition has about 0.05%, 0.2%, 0.3%, 0.4%, 0.5%, 0.6%, 0.7%, 0.8%, 1.0%, 1.2%, 1.5%, 2%, 5%, 6%, 8%, 10%, (or any % in between), from 0.1% to about 0.8%, from about 0.1% to about 1.5% w/w of the API composition.

In one embodiment, the surfactant composition comprises an anionic surfactant, cationic surfactant, zwitterionic surfactant, a non-ionic surfactant, or a combination thereof.

Example surfactants include tween 80, sodium dodecyl sulfate, alpha Tocopherol, benzalkonium chloride, benzyl alcohol, cetostearyl Alcohol, cetrimide, cetylpyridinium chloride, docusate dodium, glyceryl monostearate, glyceryl palmitostearate, linoleic acid, macrogol 15 hydroxystearate, myristic acid, myristyl alcohol, macrogol cetostearyl ether, macrogol lauryl ether, macrogol oleyl ether, macrogol stearyl ether, polyoxyethylene alkyl ethers, polyoxyethylene castor oil derivatives, polyoxyethylene sorbitan fatty acid esters, polysorbate 20, polysorbate 40, polysorbate 60, polysorbate 80, polyoxyethylene stearates, polyoxyl 40 stearate, caprylocaproyl macrogolglycerides, lauroyl macrogolglycerides, linoleoyl macrogolglycerides, oleoyl macrogolglycerides, stearoyl macrogolglycerides, polyoxylglycerides, sodium stearyl fumarate, sorbitan esters (sorbitan fatty acid esters), sorbitan laurate, sorbitan oleate, sorbitan palmitate, sorbitan stearate, sorbitan trioleate, sorbitan sesquioleate, stearic acid, stearyl alcohol, or a combination thereof. In one embodiment, the surfactant composition comprises sodium dodecyl sulfate, tween 80, tween 60, or a combination thereof.

In one embodiment, the chewable composition has from 0.1% to 10% w/w of the surfactant composition. In one embodiment, the weight ratio of the API composition and the surfactant composition is from 5 to about 20, from 8 to about 12, or any number in between.

Through extensively experimentation, Applicant discovered that the proper surfactant in proper amount significantly extends the shelf stability of the formulations when comparing to the formulation without the surfactant composition. The surfactant may also aid in the uniformity (or homogeneity) of the formulations making them suitable for disease treatment use. For example, the shelf stability of the gummy formulation may be extended from at least 1.5 times. In one embodiment, the shelf stability may be extended from about 6 to 12 months, from about 12 to about 18 months, from about 12 to over 24 months, and from about 9 months to over 36 months. In one embodiment, the shelf stability may be extended from about 2 to about 10 times. In one embodiment, the shelf stability may be extended 1.5, 2, 3, 4, 5, and 6 times.

In one embodiment, the composition may further comprise a complexing agent, wherein the complexing agent is configured to complex at least partially with the API increasing the solubility, modulating the flavor profile of the API, or both. The complexing agent may be capable of complexing with the PED5 inhibitor through coordinating, chelating, complexing, hydrogen-bonding, dipole-dipole interaction, van-der waals interaction, or a combination thereof. In one embodiment, the PED5 inhibitor complex is capable of masking and reducing the bitterness, astringent, metallic, or foul taste of the PED5 inhibitor.

In one embodiment, the PED5 inhibitor complex is capable of increasing PED5 inhibitor's solubility in aqueous matrix therefore facilitating the incorporation of the PED5 inhibitor into the aqueous gummy matrix.

In one embodiment, the complexing agent may be a cyclic glucose molecule (alpha-, beta-, gamma-cyclodextrin), cluster dextrin, maltodextrin, resistant starch, an oligosaccharide (such as inulin or soluble or non-soluble dietary fiber), a polysaccharide (such as a herbal polysaccharide), nucleic acid (DNA or RNA), an nucleotide molecule, an amino acid or its derivative thereof, a peptide, or an amid. In one embodiment, the complexing agent comprises cyclodextrin, a nucleotide, resistant starch, or a combination thereof.

In one embodiment, the complexing agent comprises protein, peptide, amide or polyamide, cluster dextrin, cyclodextrin, polydextrose, resistant starch, polyethylene glycol, polyunsaturated hydrocarbons, polyunsaturated fatty acids, mica, talc, zeolite, cellulose, plant particles, calcium carbonate, diatomaceous earth, chitosan, or a combination thereof. In one embodiment, the complexing agent comprises cyclodextrin, a nucleotide, resistant starch, an amide, a peptide, or a combination thereof.

In one embodiment, the complexing agent comprises an amide. Example amide includes without limitation such as N-acetyl glucosamine, n-acetyl galactosamine, 2-deoxy-2-aminoglucose N-acetyl, sialic acid N-acetyl, iminosugar N-acetyl, daunosamine N-acetyl, 2-deoxy-2aminogalactose N-acetyl, chitin, pectin, and amino acids.

Plant particles may be derived from various parts of a plant such as flower, fruit, seed, grain, nut, nutshell, root, leaves, or stems. In one embodiment, the plant particles comprise berry powder, nutshell powder, rice bran powder, strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, cranberry powder or blueberry powder. DNA rich plant powder may be preferred such as strawberry, which is an octoploid. In one embodiment, the complexing agent comprises strawberry DNA.

In one embodiment, the complexing agent comprises cluster dextrin or cyclodextrin. In one embodiment, the cyclodextrin comprises alpha-dextrin, beta-cyclodextrin, gamma-cyclodextrin, or a combination thereof. In one embodiment, the cyclodextrin comprises essentially gamma-cyclodextrin.

In one embodiment, the composition comprises PED5 inhibitor and cyclodextrin at a molar ratio of from about 1:1 to about 1:100, from about 1:1 to about 1:20, or any ration in between. In one embodiment, the molar ratio of PED5 inhibitor and cyclodextrin is about 1:2, 1:5, 1:8, or 1:10.

In one embodiment, the binding composition may include at least 2 binding agents selected from saccharides or sugar alcohols. Example saccharides include monosaccharide, di-saccharide, tri-saccharide, or a combination thereof. The binding composition may be sugared, low-sugar, or sugar-free.

In one embodiment, the binding composition comprises glucose, sucrose (table sugar), fructose, or a combination thereof. Sucrose may be from any source including without limitation cane, beet, coconut, or other plants.

For low-sugar or sugar free chewable composition, the binding composition may include L-fructose, L-glucose, L-galactose, allulose, sorbose, tagatose, D-maltose (1,4-diglucose), an isomer of D-sucrose (1,2-fructose glucose), trehalose, isomaltulose (isomaltuose), raffinose or a combination thereof. In one embodiment, the binding composition consists essentially of tagatose, allulose, or a combination thereof. In one embodiment, the binding composition consists essentially of tagatose, isomaltulose, or a combination thereof. In one embodiment, the binding composition consists essentially of isomaltulose, allulose, trehalose, or a combination thereof.

In one embodiment, the binding composition comprises sugar alcohol. In one embodiment, the binding composition comprises essentially sugar alcohol. In one embodiment, the binding composition comprises mannitol, sorbitol, xylitol, lactitol, isomalt, maltitol, hydrogenated starch hydrolysates (HSH), glycerol, erythritol, or a combination thereof. In one embodiment, the binding composition consists essentially of mannitol, maltitol, isomalt, or a combination thereof. In one embodiment, the binding composition consists essentially of mannitol, sorbitol, erythritol or a combination thereof. In one embodiment, the binding composition consists essentially of mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof.

In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof.

In one embodiment, the binding composition consists essentially of mannitol, fructose, isomalt, or a combination thereof. In one embodiment, the binding composition consists essentially of mannitol, N-acetylglucosamine, isomalt, or a combination thereof. In one embodiment, the binding composition consists essentially of mannitol, maltitol, sucrose, or a combination thereof. In one embodiment, the binding composition consists essentially of mannose, maltitol, xylitol, or a combination thereof. In one embodiment, the binding composition consists essentially of mannitol, sorbitol, or a combination thereof.

In one embodiment, the binding composition consists essentially of trehalose, xylitol, sorbitol or a combination thereof. In one embodiment, the binding composition consists essentially of mannitol, isomaltulose, xylitol, sorbitol or a combination thereof. In one embodiment, the binding composition consists essentially of maltitol, allulose, resistant starch or a combination thereof. In one embodiment, the binding composition consists essentially of maltitol, sorbitol, allulose, or a combination thereof. In one embodiment, the binding composition consists essentially of maltitol, tagatose, or a combination thereof. binding composition consists essentially of allulose, tagatose, maltitol, isomalt, or a combination thereof. In one embodiment, the binding composition consists essentially of isomalt, allulose, or a combination thereof. In one embodiment, the binding composition consists essentially of isomalt, sorbitol, allulose, or a combination thereof.

In one embodiment, the binding composition may comprise allulose, maltitol, or a combination thereof. The ratio of allulose and maltitol may be from about 1:1 to about 2:1, about 10:7, or about 10:6. In one embodiment, the binding composition may comprise maltitol, sorbitol or a combination thereof. The ratio of maltitol and sorbitol may be from about 1:1 to about 5:3. In one embodiment, the binding composition may comprise allulose, sorbitol or a combination thereof. The ratio of allulose and sorbitol may be from about 10:8 to about 2:1. In one embodiment, the binding composition may comprise allulose, xylitol or a combination thereof. The ratio of allulose and xylitol may be from about 10:8 to 2:1. In one embodiment, the binding composition may comprise maltitol, xylitol or a combination thereof. The ratio of maltitol and xylitol may be from about 8:10 to 3:2. In one embodiment, the binding composition may comprise allulose, erythritol or a combination thereof. The ratio of allulose and erythritol may be from about 1:1 to 2:1.

For the chewable composition that is substantially free of glucose, sucrose, and fructose, the composition may further comprise a polymer stabilizer, wherein the polymer stabilizer is water-soluble. Through significant and extensive experimentation, Applicant discovered that a stabilizing polymer significantly extends the stability of a low-sugar or sugar-free chewable composition formulations for about 1.5, 2, 3, 4, 5, 7, 8, 9, or over 10 times when compared to same formulations without the polymer stabilizer. For example, the stability may be extended from about 2 weeks to about 8 months, about 3 months to about 9 months, about 6 months to about 12 months, about 5 months to about 14 months, about 9 months to over 24 months, about 10 months to over 36 months.

The water-soluble polymer stabilizer may include a polysaccharide, a polyvinyl alcohol, a polyalcohol, a vinyl alcohol, a peptide, a cationic polymer, a polyphenol, or a combination thereof.

In one embodiment, the polymer stabilizer may be a polymer of monosaccharide monomers (i.e. polysaccharide) selected from glucose, fructose, mannose, galactose, arabinose, rhamnose, xylose, galacturonate, glucuronate, N-acetylgalactosamine, N-acetylglucosamine, or a combination thereof, and wherein the polymer comprises from about 5 to about 500 monosaccharide monomers.

In one embodiment, the polymer stabilizer may be a polysaccharide. The polysaccharide may be cationic, anionic, or nonionic. It could be homo-polysaccharide or hetero-polysaccharide. In one embodiment, the polysaccharide comprises from about 5 to about 50 monomers.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccharide, fructo-oligosaccharide, galactomannan oligomers, oligomers of ribose, xylose, arabinose, rhamnose, or a combination thereof.

In one embodiment, the polysaccharide comprises alpha-mannose monomers, beta-mannose monomers, beta-glucose monomers, or a combination thereof. In one embodiment, the polysaccharide comprises a polymer of glucose monomer or mannose monomer linked through glycosidic bonds. In one embodiment, the glycosidic bond is substantially free of 1,4-alpha-glycosidic bond. In one embodiment, the glycosidic bond comprises 1,2-alpha glycosidic bond, 1,3-alpha glycosidic bond, 1,2-beta glycosidic bond, 1,3-beta glycosidic bond, or a combination thereof.

In one embodiment, the polymer stabilizer comprises soluble fiber from tapioca, soluble corn fiber, soluble fiber from chicory root, soluble fiber from dandelion, maltodextrin, resistant maltodextrin, 6-20 β-1,4-linked glucopyranose units, 6-20 β-1,3-linked glucopyranose units, 6-20 θ-1,2-linked glucopyranose units, 6-20 a-1,3-linked glucopyranose units, 6-20 a-1,2-linked glucopyranose units, or combination thereof. In one embodiment, the polymer stabilizer comprises maltodextrin.

In one embodiment, the vinyl alcohol comprises a hydroxy methyl acrylate.

In one embodiment, the peptide comprises a collagen, a cationic peptide, or a combination thereof.

In one embodiment, the chewable composition includes from about 1% to 10%, 1% to 5%, 2% to 6%, 0.5% to 15% w/w of the polymer stabilizer. In one embodiment, the chewable composition comprises at least 2% w/w of the polymer stabilizer. In one embodiment, the chewable composition comprises from about 3% to 10%, 5% to 8%, 6% to 7% w/w of the polymer stabilizer.

In one embodiment, the binding composition consists essentially of isomalt, allulose, or a combination thereof with resistant maltodextrin as the stabilizer. In one embodiment, the binding composition consists essentially of isomalt, maltitol, or a combination thereof with polydextrose as the stabilizer.

In one embodiment, the binding composition is substantially sugar-free. In one embodiment, the binding composition is substantially free of any sugar having a glycemic index of more than 50. In one embodiment, the binding composition is substantially free of sugar alcohols.

In one embodiment, the binding composition has a glycemic index of more than 70. In one embodiment, the binding composition has a glycemic index of 220, 200, 170, 160, 120, 100, 80, or 70. In one embodiment, the binding composition has a glycemic index of less than 50, 30, 20, 15, 10, 8 or 5. In one embodiment, the binding composition has a glycemic index of 0.

In one embodiment, the chewable composition has a glycemic index of more than 70. In one embodiment, the chewable composition has a glycemic index of 220, 200, 170, 160, 120, 100, 80, or 70. In one embodiment, the chewable composition has a glycemic index of less than 50, 30, 20, 15, 10, 8, or In one embodiment, the chewable composition, having a glycemic index of about 0.

In one embodiment, the chewable composition comprises from about 50% to 85% w/w of the binding composition. In one embodiment, the chewable composition comprises from about 60% to 80%, 65% to 75%, 65% to 60%, 67% to 71% or 68% to 69% w/w of the binding composition. In one embodiment, the chewable composition comprises from about 70%, 67%, 68%, 70%, 80% w/w of the binding composition.

In one embodiment, the weight ratio of the binding composition and the polymer stabilizer may be from about 5:1 to 20:1, 6:1 to 15:1, 5:1 to 10:1, 10:1 to 12:1, 12:1 to 15:1, 10:1 to 20:1, or from 8:1 to 18:1. In one embodiment, the weight ratio of the binding composition and the polymer stabilizer from about 10:1 to 15:1.

In one embodiment, the gelling composition comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

In one embodiment, the gelling composition comprises pectin. In one embodiment, the pectin has a methoxyl content (i.e., esterification degree or DE) not less than about 15%, 20%, 40%, 50% or 65%. In one embodiment, the methoxyl content is from about 15% to 40%, 15% to 25%, 16% to 24%, 30% to 70%, 50% to 65%, 55% to 65%, 59% to 63%, or 60% to 80%.

In one embodiment, the pectin has an amide content not less than about 15%, 20%, 30%, or 40%. In one embodiment, the amid content is from about 12% to 40%, 15% to 35%, 15% to 25%, 20% to 25%, 25% to 40%.

In one embodiment, the total of the methoxyl content and the amide content is from about 36% to 70%. In one embodiment, the methoxyl content is more than about 25% and the amide content is not less than about 20. In one embodiment, the methoxyl content is from about 16% to 24% and the amide content is from about 20% to 25%. In one embodiment, the methoxyl content is from about 56% to 66% and the amide content is from about 0.1% to 0.5%.

In one embodiment, the gelling composition comprises gelatin. In one embodiment, the gelling composition comprises pectin, gelatin, collagen, or a combination thereof. In one embodiment, the gelling composition comprises pectin and collagen in a ratio from about 1:1 to about 1:3. In one embodiment, the gelling composition comprises pectin and collagen in a ratio about 1:2.

In one embodiment, the chewable composition includes from about 0.5% to 10%, 1.5% to 2.5%, 0.5% to 1.5% w/w of the gelling composition. In one embodiment, the gelling composition may further comprise carrageenan. In one embodiment, the chewable composition includes from about 5% to 10% w/w of the gelling composition, wherein the gelling composition comprises gelatin.

In one embodiment, the chewable composition may further include an herb composition, an antioxidant composition, a vitamin composition, a mineral composition, an amino acid composition, a probiotics composition, or a prebiotics composition.

In one embodiment, the herb composition comprises ginger, prickly ash bark, turmeric, motherwort, eleuthero, any ginseng variety including without limitation American ginseng, panex ginseng, or red panax ginseng, ashwagandha, Schisandra, wild oats, passion flower, Valerian, Chinese yam (Dioscorea sp), Eucommia (Eucomnia ulmoides), Ginkgo Biloba, Deer antlers (Cervi pantotrichum), Seahorse (Hippocampus kelloggii), Tribulus terrestris, Tongkat Ali (Eurycoma longiflora), Gambir, Muira puama (Ptychopetalum olacoides), Yohimbe (Pausinystalia yohimbe), Epimedium (Horny Goat Weed), Maca (Lepedium meyenii W), Oatstraw, Catuaba (Erythroxylum catuaba), Nettle leaf (Urtica dioica), Dadder seed (Tusizi), Cordyceps (Dongchongxiacao), Cayenne, Astragalus, Sarsparilla (Smilax officinalis), Licorice root, Pumpkin seed, Yohimbe, Eurycoma longifolia, beet, extract, powder, isolate or distillate thereof.

In one embodiment, the antioxidant composition comprises Vitamin E, Vitamin C, beta-carotene, gallic acid, selenium, selenium yeast, phenolics, anthocyanins, flavonoids, polyphenols, whey, bioflavonoids, theobromine, anthracenes, carotenoids, lutein, zeaxanthin, ginko biloba, berry extract, resveratrol, saffron, Sangre de grado (dragon's blood), cocoa, or derivatives thereof.

In one embodiment, the vitamin composition comprises vitamin A, B, C, D, E, K or a combination thereof. In one embodiment, the vitamin composition comprises Vitamin B9 (or folic acid), Vitamin D, Vitamin B3 (Niacin or Niacinamide), Vitamin C, or a combination thereof.

In one embodiment, the mineral composition comprises salts of calcium, iron, zinc, magnesium, sodium, chloride, potassium, copper, molybdenum, manganese, phosphorus, iodine, nickel, boron, or selenium, or a combination thereof. In one embodiment, the mineral composition comprises zinc citrate, zinc gluconate, zinc sulfate, zinc acetate, boron citrate, or a combination thereof.

The amino acid may be natural or non-natural occurring. In one embodiment, the amino acid composition comprises histidine, a branched chain amino acid, leucine, iso-leucine, valine, L-5 hydroxytryptophan (5-HTP), an essential amino acid, histidine, lysine, methionine, phenylalanine, threonine, tryptophan, L-theanine, beta-alanine, L-arginine, citrulline, carnitine, alanine, propionyl-L-carnitine, or its derivative thereof. In one embodiment, the amino acid composition comprises L-arginine, citrulline, or its derivative thereof.

In one embodiment, the prebiotic composition comprises gum arabic, chicory root powder or extract, wheat bran powder or extract, acacia gum, guar gum, Artichoke fiber, oat fiber, soluble corn fiber, inulin, resistant maltodextrin, resistant starch, or a combination thereof. In one embodiment, the probiotic composition comprises Lactobacillus acidophilus, Lactobacillus rhamnosus GG, Saccharomyces boulardii, Bifidobacterium bifidum, Bacillus coagulans, or a combination thereof.

In one embodiment, the chewable composition may further include an additive selected from sweeteners, food acids, flavoring agents, coloring agents, humectants, bulking agents, fatty acids, triglycerides, plasticizers, thickeners, preservatives, or and a mixture thereof.

In one embodiment, the sweetener comprises artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N—[N-[3-(3-hydroxy-4-methoxyphenyl)propyl]-L-.alpha.-aspartyl]-L-phenylalanine 1-methyl ester, N—[N-[3-(3-hydroxy-4-methoxyphenyl)-3-methylbutyl]-L-alpha-aspartyl]-L-phenylalanine 1-methyl ester, N—[N-[3-(3-methoxy-4-hydroxyphenyl)propyl]-L-alpha-aspartyl]-L-phenylal-anine 1-methyl ester, salts thereof, licorice or its extracts or isolates, or a mixture thereof.

In one embodiment, the chewable composition is substantially free of artificial sweeteners, non-sugar sweeteners or sugar substitutes.

In one embodiment, the chewable composition may further include a coating composition. In one embodiment, the coating composition comprises isomalt, allulose, tagatose, xylitol, erythritol, sorbitol, mannitol, or a combination thereof. In one embodiment, the coating composition may have a particle size from about 0.6 mm to about 0.75 mm. In one embodiment, the coating composition may have a particle size of about 400 microns.

In one embodiment, the chewable composition may include the binding composition comprising maltitol and at least one of allulose, xylitol, erythritol, maltitol, sorbitol, and mannitol, and the coating composition including maltitol.

The pH of the chewable composition may be from about 3 to 5, 2 to 6, 2 to 4, 3 to 4, 2 to 5, or any number in between. In one embodiment, the pH of the composition is less than 5. In one embodiment, the pH of the composition is more than 7. In one embodiment, the pH of the composition is about 9.

In one embodiment, the gelling composition comprises pectin. In one embodiment, the pH of the composition is less than 3, 3.5, or 4. In one embodiment, the pH of the composition is more than 3 or 3.5. In one embodiment, the pH of the composition is more than 4 or 4.5. In one embodiment, the pH of the composition is more than 7. In one embodiment, the pH of the composition is about 9.

In one embodiment, the gelling composition comprises gelatin. In one embodiment, the pH of the composition is more than 6.

In one embodiment, the chewable composition may include the API composition comprises tadalafil or a derivative thereof, the surfactant comprises tween 80, sodium dodecyl sulfate, the complexing agent comprises cyclodextrin, wherein the PED5 inhibitor and the complexing agent have molar ratio from about 1:1 to about 1:10. In one embodiment, the binding composition comprising maltitol, mannitol and isomalt and the polymer stabilizer comprises polydextrose soluble fiber, wherein the binding composition and the polymer stabilizer have a w/w ratio from about 20:1 to about 4:1, from about 10:1 to 5:1, from about 15:1 to about 10:1, from about 8:1 to about 4:1. In one embodiment, the binding composition may include maltitol and allulose.

In one embodiment, the chewable composition comprises from about 2 mg to about 20 mg tadalafil and from about 0.1% to about 1% sodium dodecyl sulfate or tween 80. The binding composition comprises glucose, sucrose and optionally fructose.

In one embodiment, the chewable composition comprises from about 5 mg, 10 mg, 20 mg or 40 mg tadalafil, from about 0.1% to about 0.5% sodium dodecyl sulfate or tween 80 with a polymer stabilizer. In one embodiment, the binding composition comprises sugar alcohol. In one embodiment, the binding composition comprises allulose, tagatose, a sugar alcohol or a combination thereof. The polymer stabilizer comprises polydextrose, soluble corn fiber, soluble tapioca fiber, inulin or beta-glycan.

In another aspect, the application provides methods of making the chewable composition. In one embodiment, the method includes the steps of dividing the binding composition into a first binding portion and a second binding portion, combining a first mixture and water and heating to at a first elevated temperature to provide a first solution, wherein the first mixture comprises the first binding portion and optionally the polymer stabilizer, combining the second mixture and water at a second elevated temperature to provide a second solution, wherein the second mixture comprises the gelling composition with the second binding composition that is equal to, twice, three time, or four times the mass of the gelling composition, and the complexing agent, mixing the second solution into the first solution at a third elevated temperature to provide a third mixture, wherein the third mixture has a Brix number from about 80 to about 85 or from about 78 to about 86, adjusting pH of the third solution with a buffer salt to from about 3 to about 7.

In one embodiment, the method may further include the step of adding coloring agent, flavoring agent, or a combination thereof into the third mixture to provide a molding mix having a Brix from about 78 to about 86.

The API composition and surfactant composition may be added to the second mixture, the third mixture, or the molding mixture. In one embodiment, the API composition and surfactant composition may be added to the third mixture together with the coloring agent or flavoring agent.

In one embodiment, the method may further include the step of adding the molding mix to a preformed shaped cavity. In one embodiment, the method may further include the step of cooling the molding mix (or mixture) in the preformed shaped cavity until the molding mixture forms into the chewable composition piece.

The first, second and third elevated temperature may be independently from about 175° F. to about 275° F., from 175° F. to about 200° F., from 170° F. to about 210° F., or any temperature in between.

In one embodiment, the method comprises the following steps. In a first container, a gelling composition is mixed with a portion of a binding composition. Optionally, a buffering salt may be added. The components are mixed until homogeneous to provide a first mixture (Mix 1). In one embodiment, the gelling composition comprises pectin. In one embodiment, the portion of the binding composition comprises sorbitol and isomalt, or allulose and maltitol. In one embodiment, the buffering salt may include sodium citrate, potassium citrate, or a combination thereof.

In a second container, the remaining portion of the binding compositions added. In one embodiment, the remaining portion of the binding composition comprises sorbitol, isomalt, and mannitol. The components are mixed until homogeneous to provide a second mixture (Mix 2).

In a third container, a food acid is dissolved in an aqueous solution with additives such as coloring agent and flavoring agent. In one embodiment, the food acid comprises citric acid, malic acid, acetic acid, or a combination thereof. In one embodiment, the aqueous solution comprises water, ethanol, glycerol, or any combination thereof. All components are mixed and warmed until a homogenous solution is achieved to provide a third mixture (Mix 3). In one embodiment, the components are warmed to 175° F.

To a first reaction container, the API composition and the surfactant may be added. Optionally, the complexing agent may be added. In one embodiment, the API composition comprises tadalafil or a derivative thereof. In one embodiment, the complexing agent comprises beta-cyclodextrin. Water is then added to the first reaction container. In one embodiment, the water may be heated first before adding to the first reaction container. In one embodiment, the water is heated to at least 200° F.

Then the Mix 1 is added to the first reaction container with stirring to provide a first solution. The mixture is stirred until the gelling composition fully swells and disperses. In one embodiment, the first solution may be brought to a light boil.

To a second reaction container, Mix 2 is added, followed with addition of water. The components are mixed to provide a second solution. In one embodiment, the second solution may be brought to a boil. Then, the first solution is combined with the second solution with mixing. The mixture is heated to a Brix number of at least 82 Brix. Then, Mix 3 is added dropwise with stirring to provide a molding mix having a Brix number of at least 82 Bix.

The molding mix was then added to a mold to provide individual gummy pieces. The mold may be a silicon mold, a starch mold, or a sugar alcohol mold. In one embodiment, the sugar alcohol mold is made by compacting a sugar alcohol composition powder or particles in a container to create a compacted an sugar alcohol mass, and stamping the compacted mass with a desirable shape to create mold cavities in the compacted mass. In one embodiment, the sugar alcohol composition comprises maltitol, isolmalt, or a combination thereof.

The molding mix may be injected or deposited into the mold cavities to form gummy pieces. The gummy piece may be any shape and size. The formed chewable composition may be in square shape, gumdrop shape, hexagon shape, partial ball shape, animal shape, cartoon shape, or any desirable shape. The shape may be hexagon, square, half ball, gumdrop, heart, bear, or any other shapes.

The formed composition may be from about 1 g to about 10 g, from about 2 g to about 7 g, from about 3 g to about 5 g, or any number in between. In one embodiment, the gummy piece may have a weight from about 3 g, 4 g, 5 g, 6 g, 7 g, 7.5 g, to about 8 g.

Each gummy piece may contain from about 2 mg to about 500 mg of PED5 inhibitor. In one embodiment each gummy piece may contain about 25 mg, 50 mg, 100 mg or 150 mg sildenafil, its derivative or salt thereof. In one embodiment, each gummy piece may contain about 2 mg, 5 mg, 10 mg, 20 mg, 40 mg, or 100 mg tadalafil. In one embodiment, each gummy piece may contain about 2 mg, 5 mg, 10 mg, 30 mg, 40 mg of vardenafil. In one embodiment, each gummy piece may contain 25 mg, 50 mg, 100 mg, 200 mg, or 300 mg of avanafil.

The gummy piece may be further coated with a coating composition. The coating composition may prevent gummy piece to stick with each other. The coating composition may include isomalt, maltitol, or other low glycemic sugar or sugar alcohol. In one embodiment, the coating composition comprises isomalt. In one embodiment, the coating composition comprises maltitol.

Through extensive experimentation, the Applicant discovered that the water solubility property of the coating composition is critical in maintaining gummy formulation stability. In one embodiment, the coating composition may have a water solubility of at least 2000 g/L, 1750 g/L, 1500 g/L, 1000 g/L, 500 g/L of water at room temperature. In one embodiment, the water solubility of the coating composition is at least 1500 g/L. In one embodiment, the coating composition comprises isomalt, allulose, maltitol, maltodextrin, inulin, starch, bran, xylitol, sorbitol, tagatose, erythritol, or a combination thereof.

In a further aspect, the application may provide methods for treating a condition method for treating erectile dysfunction in a subject, benign prostatic hyperplasia in a subject, or pulmonary arterial hypertension in a subject or comprising administrating an effective amount of the chewable composition disclosed herein to the subject.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the chemical structure of tadalafil;

FIG. 2 shows, from top to bottom, representative non-ionic, anionic, cationic, and zwitterionic surfactants;

FIG. 3 shows the capture of tadalafil by the hydrophobic interior of a micelle;

FIG. 4 shows the chelation of a drug by cyclodextrin;

FIG. 5 shows a representative amide resonance; and

FIG. 6 shows the association of tadalafil with N-acetylglucosamine through the amide moiety, in which the nitrogen with a partial positive charge associates with the oxygen with a partial negative charge, the association of the two amide groups results in a six-member ring, one association is shown for clarity and either one (on either amide tadalafil amide) or two associations are possible.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

As used herein, “semi-solid chewable gel composition,” “chewable composition” and “gummy composition” are used interchangeably.

As used herein, “allulose” and “psicose” are used interchangeably.

As used herein, “isomaltulose” and “palatinose” are used interchangeably.

As used herein, “mix” and “mixture” may be used interchangeably.

In one embodiment, the chewable composition comprising an active pharmaceutical ingredient (API) composition comprising a PED5 inhibitor, a surfactant composition, wherein the surfactant composition and the API composition have a weight ration from about 0.1 to about 15, a binding composition, comprising a mono- or di-saccharide, a sugar alcohol, an oligosaccharide, or a combination thereof, and a gelling composition in a sufficient amount to provide a cohesive gelled product.

Active Pharmaceutical Ingredients (APIs)

In one embodiment, the API composition comprises tadalafil, sildenafil, vardenafil, avanafil, or a combination thereof. In one embodiment, the API composition comprises tadalafil. In one embodiment, the API composition comprises sildenafil. In one embodiment, the chewable composition comprises from 0.16% to 0.63% w/w of the API composition.

Gummy can be used as an efficient delivery mechanism for an API. Firstly, gummy composition is easy to consume especially for patient populations having swallowing difficulty including without limitation pediatric and geriatric population. Second, gummy, if formulated to taste good, increases patient's compliance in taking medication. Third, when being consumed, gummy composition dissolves and coats the mucosal membrane allowing for direct absorption of APIs through a trans-mucosal process, potentially bypassing the digestion track and reducing first-pass effect.

A good tasting gummy product has the advantage of being palatable and convenient to consume, leading to increased patients' compliance in taking the medication. The act of chewing and dissolving the gummy allows for trans-mucosal absorption of the API directly into blood circulation for fast action and bypassing the liver and avoiding first-pass effect.

Gummy has not been used for the delivery of active pharmaceutical ingredient(s) (API). One reason is that many APIs are extremely foul or bitter in taste. Tadalafil is one example. The inclusion of the API may lead to a foul-tasting product. Hence, pharmaceuticals have typically been reserved for non-chewable items that make for minimal mouth contact. Thus, a major advancement in API delivery is a delivery mechanism that allows chewing and mouth contact with little to none of the bitterness or medicinal taste of the API. This disclosure gives a delivery mechanism for the PED5 inhibitor that eliminates the bitterness and medical taste of the API allowing the formulation to be chewed.

Tadalafil is a type-V phosphodiesterase (PDE-5) inhibitor that increases blood flow to genital parts of the body. Tadalafil is indicated for erectile dysfunction, sexual dysfunction, and certain cardiac symptoms. Tadalafil is also known as Cialis. Tadalafil is bitter and unpleasant tasting.

In one embodiment, the gummy formulation for the delivery of tadalafil is presented. The gummy delivers 2.5, 5, 10 20 mg, or 40 mg of tadalafil in a format that has a pleasing taste. In one embodiment, the gummy consists of a gelling agent, sugars, water, and complexing agents that chelates tadalafil. Tadalafil is coordinated in the gummy structure which leads to a reduction or elimination of the unpleasant perception of the tadalafil API. The tadalafil gummy allows for fast absorption and faster relief of symptoms.

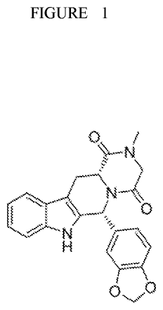

The chemical structure for tadalafil is shown in FIG. 1. The IUPAC name for tadalafil is 2R,8R)-2-(1,3-benzodioxol-5-yl)-6-methyl-3,6,17-triazatetracyclo[8.7.0.0^{3,8}.0^{11,16}]heptadeca-1(10),11,13,15-tetraene-4,7-dione.

Tadalafil functions by increasing the level of the cyclic guanosine-3,5-monophosphate (cGMP), which is an important secondary messenger that controls many physiological processes. The level of intracellular cGMP is determined by the activities of the cyclase enzyme which produces it, and the type-V phosphodiesterase (PDE-5) that degrades it. The inhibition of PDE-5 increases the level of cGMP, and therefore can be used in a therapeutic strategy for male erectile dysfunction, and also for the treatment of cardiovascular diseases.

Surfactants

Applicant discovered that surfactants are useful with improving flavor and stability of tadalafil gummy formulation. Surfactants are molecules with amphiphilic properties. Surfactants form micelles when added to water. The interior of the micelle is hydrophobic whereas the exterior is hydrophilic. Micelles have the ability to dissolve hydrophobic materials and suspend them in water forming emulsions.

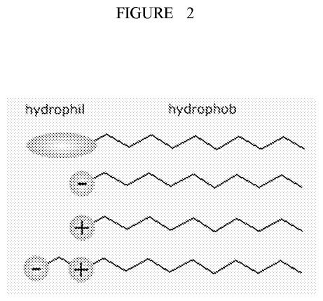

There are many types of surfactants: cationic, anionic, zwitterionic, or non-ionic. Cationic examples include ammonium or pyridinium; anionic examples include carboxylates, sulfonates, sulfates, and phosphates; zwitterionic examples include tetraalkyl ammonium carboxylates, tetraalkyl ammonium sulfates, and ammonium phosphates, while non-ionic include polyethers, and alcohols. The concept of surfactants is shown in FIG. 2.

One problem that needs to be solved for using surfactants is the pH of the gummy. Gummies are typically acidic and have a typical pH range from a pH of 2.5 to pH of 4. If the pKa of the surfactant is higher that the pH of the gummy then the surfactant may become non-functional and precipitate. For example, the pKa of stearic acid, of which sodium stearate is a surfactant, is 4.75. At a pH above the pKa the sodium stearate surfactant is ionized and functions well, but below a pH below the pKa the stearate is protonated to stearic acid which is insoluble in water and the surfactant precipitates. Thus, the pKa of the surfactant needs to be below the acidity of the gummy formulation. In one embodiment, the surfactants are sulfonates and/or sulphate surfactants since their pKa's are typically below 0. The phosphate surfactants with pKa is below 2 can be useful. Non-ionic surfactants and zwitterionic surfactants can also be useful if they are stable in a high ionic strength medium.

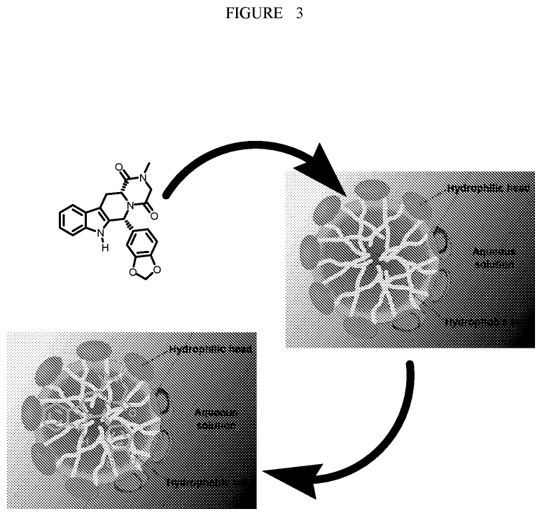

Surfactants are particularly well suited to capturing molecules inside their micelles; especially hydrophobic molecules such as Tadalafil. Applicant found that the addition of a surfactant helps to solubilize tadalafil into water and make it more accessible to the cyclodextrin molecules to chelate. FIG. 3 illustrates the process of tadalafil uptake by micelles formed from surfactants. While tadalafil is practically insoluble, there is a small, mol fraction solubility (5.74·10E-7 at 298.15 K) in water. The tadalafil that solubilizes is captured by the micelles. By Le Chatelier's principle the capture of tadalafil by micelles leads to more tadalafil being solubilized by water which in turn leads to more capture by micelles. In this way the micelles help to dissolve the tadalafil.

The micelles help the flavor profile and stability of tadalafil gummies. In initial water solution the tadalafil is surrounded by the surfactant, which keeps the drug away from the taste buds on the tongue. The surfactant can aid in the transfer of tadalafil to the complexing agent, such as cyclodextrin, as the gummy becomes increasingly rich in carbohydrates during the formation process.

Complexing Agents

Complexing agents may be useful to complex with API masking or modulating flavor profile, reducing bitterness, increasing the solubility of the API, or increasing the stability of the formulation. In one embodiment, the complexing agent may form an inclusion complex with API. In one embodiment, the complexing agent comprises cyclic glucose (i.e. cyclic dextrin).

Cluster dextrin have a ring structure with many branches of long chains of glucose units pendent to the ring forming a helical structure. The helical structure along with the ring structure of cluster dextrin are both able to chelate small molecules such as, at least partially, tadalafil. The chelation takes place by the aromatic rings of tadalafil fitting inside the helical structure.

There are three major forms of cyclodextrin: alpha, beta, and gamma. Alpha cyclodextrin consists of a ring of 6 glucose units while beta has 7 glucose units in a ring and gamma has 8 glucose units in a ring. The ring structures form a crown. The inside of the crown is able to chelate small molecules; especially hydrophobic molecules.

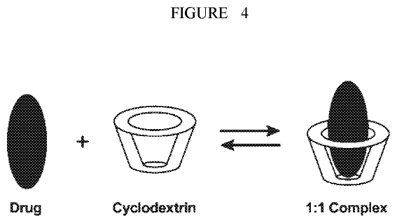

The chelation of a drug by cyclodextrin is shown in FIG. 4. The aromatic group of tadalafil may fit inside the cyclodextrin ring structure. The inside cavity of the cyclodextrin structure is largely hydrophobic, which is favorable for complexing aromatic systems that are also hydrophobic. The formation of the chelate structure is endothermically favorable due to electrostatic interactions of the p system of the aromatic moiety within the hydrophobic cavity and electronic interaction with the hydrogen atoms and glycidyl ether bonds. It is these electronic interactions between these systems of the cyclodextrin and the pi system that gives the favorable heat of formation. The alpha, beta, and gamma cyclodextrins do not form the complex with tadalafil equally. Tadalafil forms stable complexes with all the cyclodextrins with gamma being the most stable complex and alpha being the least stable.

The coordination of tadalafil by cluster dextrin molecules is more by chance therefore the complexing efficiency may be low. Cluster dextrin molecules have a broad range of cyclic ring and helical structures. Statistically there are some cyclic and helical structures that meet the criteria for chelation of tadalafil.

An effective method of flavor mitigation for an API that has functionality similar in structure to nucleotide bases is the addition DNA or RNA or sources rich in RNA and DNA. The base structures on DNA consists of two purines and two pyrimidines. The purines are adenine and guanine while the two pyrimidines are cytosine and thymine.

A chemical structure such as tadalafil which has a pyrimidine-like structural unit will be naturally attracted and bind the purine bases in DNA thereby complexing the API and reducing its contribution to the overall flavor of the gummy. Foods that are particularly high in DNA are the fruits and vegetables and their powders. Fruits and vegetables have many strands of DNA and RNA that go beyond the normal helix (diploid). The strawberry is famous for being particularly rich in DNA. Each cell in a strawberry contains 8 copies of its genetic information (octoploid), while most animal cells only contain 2 copies (diploid). Other examples of high-density DNA and/or RNA are the sweet potato (hexaploid), sugar cane (octoploid), apple (triploid), peanut (tetraploid), and kumquats (tetraploid). Any polyploidy plant material can be used as the DNA/RNA source.

In one embodiment, the chewable composition further includes a complexing agent that coordinates (or complexes) with tadalafil. The complexing agent may be a cyclodextrin, a cluster dextrin, a nucleotide, a nucleic acid, or N-acetylglucosamine. Through extensive research, Applicant discovered with surprise that N-acetylglucosamine works to help reduce the bitterness or unpleasant flavors as well as the surfactant and cyclodextrin. The result is a tadalafil gummy that has excelling chew, taste, and flavor profile.

N-Acetylglucosamine is an amino sugar where the hydroxy moiety on carbon 2 has been substituted with an amino moiety and subsequently amidated with acetic acid. Amides have the chemical structure

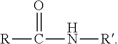

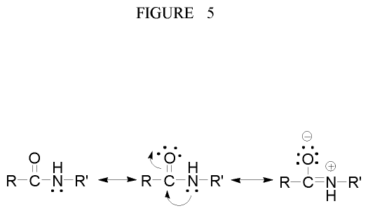

The chemical structure is such that there is an electron rich group, containing the nitrogen, adjacent to an electron deficient group, the carbonyl which contains the carbon and oxygen. Electron density can be transferred from the nitrogen to the carbonyl to form a resonance structure as shown in FIG. 5.

The resonance structure is a Zwitterionic form of the amide structure where there is a positive charge on the nitrogen, a negative charge on the oxygen and a double bond between the nitrogen and carbon atoms.

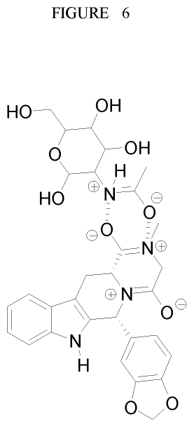

Tadalafil is also an amide, in fact, tadalafil has two amide structures. The amide structures that exist for N-acetylglucosamine also exist for tadalafil. The N-acetylglucosamine is able to associate with the tadalafil though the amide group as shown in FIG. 6. The association of N-acetylglucosamine with tadalafil through the amide moiety contributes to the reduction in the bitter medicinal taste.

Binding Composition

Binding composition binds the chewable (gummy) together through interaction with the gelling composition. The interaction may be through hydrogen bonding or through covalent bonding. In one embodiment, the binding composition comprises sugars, sugar derivatives, sugar alcohols, or a combination thereof. The binding composition may keep texture of the product soft by acting as a humectant.

In one embodiment, the binding composition comprises sucrose, glucose, fructose, or a combination thereof. In one embodiment, the chewable composition comprises from about 50% to about 90%, from about 60% to about 85% of the binding composition.

In one embodiment, the binding composition comprises a mono- or di-saccharide (i.e., sugar) having a glycemic index of less than 35, 30, 25, 20, 15, or 10, a sugar alcohol, or a combination thereof. In one embodiment, the chewable composition is substantially free of sugar having a glycemic index of more than 35. In one embodiment, the semi-solid chewable composition has a glycemic index of not more than 8, 10, 15 or 20.

In one embodiment, the chewable composition is substantially free of sucrose, fructose, glucose, sugar alcohol, sugar substitute, or non-sugar sweetener. Example sugar substitutes include, without limitation, sucralose, stevia extract or derivatives, monk fruit extract, licorice extract or derivative, tamarind extract or derivative, or their derivative thereof.

In one embodiment, the binding composition comprises a low GI sugar having a glycemic index (GI) of not more than 18, 20, 30 or 35. In one embodiment, the low GI sugar comprises allulose, sorbose, tagatose, trehalose, isomaltulose, raffinose, or a combination thereof. In one embodiment, the binding composition comprising tagatose, allulose (also known as allulose), sorbose, isomaltulose, trehalose (also known as mycose), mannose, maltose, ribose, xylose, tetroses, pentoses, hexoses, heptoses, their acid forms or a combination thereof. In one embodiment, the binding composition consists essentially of allulose, isomaltulose, and a third low GI sugar selected from a group consisting of trehalose, sorbose, tagatose, or a combination thereof. In one embodiment, the binding composition further comprises N-acetyl glucosamine.

Through extensive experimentation, processes are developed to used allulose, sorbose, tagatose, isomaltulose, trehalose in the gummy formulation allowing these sugars behave like conventional sugars such as sucrose and fructose but without the caloric significance of the convention sugars.

In one embodiment, the binding composition comprises allulose, trehalose and isomaltulose. In one embodiment, the binding composition comprises more than 20% isomaltulose. In one embodiment, the binding composition comprises from 15% to 35% isomaltulose. In one embodiment, the binding composition comprises not more than 75% of allulose. In one embodiment, the binding composition comprises from 45% to 60% allulose. In one embodiment, the binding composition comprises not more than 45% of trehalose.

In one embodiment, the binding composition comprises allulose and tagatose. In one embodiment, the binding composition comprises not more than 50% tagatose. In one embodiment, the binding composition comprises from 30% to 45% tagatose. In one embodiment, the binding composition comprises not more than 70% of allulose.

In one embodiment, the binding composition comprises isomaltulose and tagatose. In one embodiment, the binding composition comprises from 30% to 60% tagatose. In one embodiment, the binding composition comprises not more than 70% of isomaltulose.

Sugar alcohols are sweet, non-cariogenic, not digestible and provide fewer calories than many sugars. In some embodiments, sugar alcohols may mask other flavors. For example, mannitol may be used to mask bitterness Mannitol masks bitterness by a mechanism that involves the endothermic nature of mannitol dissolving into water.

In one embodiment, the binding composition comprises essentially of sugar alcohols. Example sugar alcohols include sorbitol, mannitol, erythritol, xylitol, isomalt, maltitol, lactitol, and hydrogenated starch hydrolysates. In one embodiment, the binding composition comprises mannitol, maltitol, or isomalt. In one embodiment, the binding composition comprises mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof. In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof. In one embodiment, the binding composition consists essentially of mannitol, sorbitol, or a combination thereof. In one embodiment, the binding composition consists essentially of mannitol, sorbitol, erythritol or a combination thereof.

Erythritol ((2R,3S)-Butan-1,2,3,4-tetrol) is a non-caloric polyol with a moderate sweetness of 60-80% of sucrose. Erythritol exhibits a cooling effect due to its negative heat of solution. Erythritol can improve the mouth feeling and mask certain unwanted aftertastes such as astringency and the irritant effect of intense sweeteners.

Sorbitol is highly soluble in water and is an excellent humectant. Xylitol not only non-cariogenic, it actually prevents tooth decay. Lactitol has about 30-40% of sugar's sweetening power. Its taste and solubility profile resemble sugar. Isomalt is 45-65% as sweet as sugar and does not tend to lose its sweetness or break down during the heating process. Isomalt absorbs little water. Maltitol is 75% as sweet as sugar and provides about 2-3 kcal/g. It gives a creamy texture to the formulation.

In one embodiment, the binding composition may include hydrogenated starch hydrolysates (HSH). HSH is a form of sugar alcohol from the hydrogenation of starch. The hydrogenation process yields a mixture of several sugar alcohols. HSH are nutritive sweeteners that provide 40-90% of the sweetness of sugar.

Gelling Composition

In one embodiment, the gelling composition comprises pectin, gelatin, or a combination thereof. In one embodiment, gelatin may be combined with other hydrocolloids—pectin, agar, starch, gum Arabic—to create desired textures. In one embodiment, gelatin may be combined with gum arabic as the gelling composition.

In one embodiment, the gelling composition comprises starch such as amylose starch or modified starch. There are a variety of starch modification techniques, such as contacting starch with acid, sodium or potassium hydroxide, or oxidizing the starch.

In one embodiment, the gelling composition comprises agar. Agar may be combined with locust bean gum as a gelling composition. Locust bean gum helps to prevent weeping of agar gels. The two polysaccharides from agar and locust bean gum synergistically interact with each other to form a strong gel that does not weep.

In one embodiment, the gelling composition comprises carageenans. Carrageenans or carrageenins are linear sulfated polysaccharides. Kappa-carrageenan has one sulphate group per disaccharide and forms strong, rigid gels in the presence of potassium ions. In one embodiment, locust bean gum may be used with kappa-carrageenan to prevent weeping. Gels formed from kappa-carrageenan and potassium ions are thermally reversible.

In one embodiment, the gelling composition comprises alginic acid or alginate. Alginate may form strong hydrogels when crosslinked with calcium ions.

Polymer Stabilizer

The polymer stabilizer serves to stabilize the chewable composition. In one embodiment, the polysaccharide may be water soluble.

In one embodiment, the polymer stabilizer comprises a polysaccharide of mono- or di-saccharide monomers. In one embodiment, the monomers may include glucose, fructose, mannose, galactose, arabinose, rhamnose, xylose, galacturonate, glucuronate, N-acetylgalactosamine, N-acetylglucosamine, or a combination thereof. The polysaccharide comprises from about 5 to about 50 monosaccharide monomers.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccahride, fructo-ligosaccharide, galactomannan oligomers, oligomers of ribose, xylose, arabinose, rhamnose, hyaluronic acid, or a combination thereof.

Maltodextrin is an oligiosaccharide consisting of 6-20 glucose units. Maltodextrin can be in digestible or resistant forms. Digestible maltodextrin consists of a chain of 6-20 glucose units connected by alpha-1,4-glycosidic bonds. Resistant maltodextrin consists of a chain of 6-20 glucose units connected through alpha-1,2, alpha-1,3, beta-1,2, beta-1,3, and/or beta-1,4 glycosidic bonds. As the name implies, resistant maltodextrin resists digestion as the enzyme amylase is inactive on the bonds that constitute resistant maltodextrin whereas amylase is active on the bonds that constitute maltodextrin. Maltodextrin provides for 4 Calories per gram whereas resistant maltodextrin provides for 1.6 Calories per gram. Applicant discovered that the inclusion of roughly 5% w/w maltodextrin can help stabilize the gummy products against crystallization.

In one embodiment, the polymer stabilizer may include a polysaccharide derived from an herb. In one embodiment, the polysaccharide may derive from Cistanche deserticola, Astragalus membranaceus, Rubia cordifolia, Nerium indicum, Adhatoda vasica, Withauia somnifera, and Glycyrrhiza glabra, aloe vera, Bletilla striata, Kobjar, Goji berry, elderberry, or a combination thereof.

In one embodiment, the polymer stabilizer may include a mushroom polysaccharide. In one embodiment, the mushroom polysaccharide may derive from schizophyllum commune (Schizophyllum commue), Brazilian mushroom (Agarics blaze), Cordyceps sinensis (Cordyceps sinensis), glossy ganoderma (Ganoderma lucidum), rainbow conk (Coriolus versicolor), camphor tree sesame (Anthodia camphorate), Phellinus (Phellinus linteus), coral mushroom (Pleurotus citrinopileatus), mushroom (Lentinula edodes), Liu Songgu (Agrocybe aegerita), Hericium erinaceus (Hericium erinaceus), pleurotus eryngii (Pleurotus eryngiig), petal fine and soft (Sparrasis crispa), black fungus (Auricularia auricula), Asparagus (Flammulina velutipes) or a combination thereof. In one embodiment, the mushroom polysaccharide may include chitin, hemicellulose, α- and β-glucans, mannans, xylansand, or galactans. In one embodiment, the mushroom polysaccharides may include β-glucan polymers, with the main chain consisting of β-(1→3) linkages with some β-(1→6) branches as well as chitin, mannans, galactans, and xylans.

In one embodiment, the semi-solid composition may include from about 5% to about 15% of polymer stabilizer. In one embodiment, the ratio of the binding composition and the polymer stabilizer is from about 8:1 to about 20:1.

Coating Composition

The gummy pieces or semi-solid chewable composition disclosed herein may be coated with a coating composition. In one embodiment, the coating compositions may include sugar, sugar alcohol, or a combination thereof.

In one embodiment, the coating composition may include sucrose (sanding sugar).

For low sugar or sugar free composition, example sugars may include allulose, sorbose, tagatose, trehalose, and isomaltulose, or a combination thereof. Example sugar alcohols may include erythritol, sorbitol, mannitol, maltitol, isomalt, xylitol, or a combination thereof. In one embodiment, the coating composition comprises resistant starches, fibers, inulin, or a combination thereof in one embodiment, the coating composition may include matitol, isomalte, allulose, or a combination herein.

The chewable composition may further comprise an herb composition, an antioxidant composition, a vitamin composition, a mineral composition, an amino acid composition, a probiotics composition, or a prebiotics composition.

Herbal Composition

Herbal extract or derivatives useful for the application may include flavanoids, allied phenolic compounds, polyphenolic compounds, terpenoids, alkaloids, sulphur-containing compounds, polysaccharides, flavone, flavonoids, quinone, or combinations thereof. In one embodiment, the herbal composition may comprise an adaptagen.

Antioxidants

Antioxidant may include astaxanthin, carotenoids, coenzyme Q10 (“CoQ10”), flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof.

Vitamins

Vitamins may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D, vitamin E, vitamin K, K1 and K2 (i.e., MK-4, MK-7), folic acid, biotin, choline, or combinations thereof.

Minerals

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

Amino Acids

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, carnitine, its derivative, or combinations thereof. In one embodiment, the amino acid may be a branched-chain amino acid. In one embodiment, the amino acid may be a natural amino acid. In one embodiment, the amino acid may be a non-natural amino acid.

Taurine, or 2-aminoethanesulfonic acid, is an amino acid that is widely distributed in animal tissues. It is a major constituent of bile and can be found in the large intestine, and accounts for up to 0.1% of total human body weight. Taurine can reduce bitterness by 50% when added at a concentration of 300 mM.

Probiotics

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects.

Probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

The chewable composition may contain probiotic strains in an amount ranging from 10⁵ and 10¹² cfu/g. In one embodiment, the chewable comprises between 10⁷-10¹⁰ cfu/g.

Prebiotics

Useful prebiotics are Lactose, Inulin, Fructo oligosacccharides, Galacto oligosaccharides and Xylo oligosaccharides. Prebiotics are naturally found plenty in certain fruits like bananas, asparagus, garlic, tomato and onion wheat. The characteristic features of ideal prebiotics are as follows. They are neither to be hydrolysed nor absorbed by mammalian enzymes or tissues. They selectively enrich beneficial bacteria. The most important characteristic feature is that prebiotics can alter the intestinal micro-flora and its activities.

Prebiotics when combined with probiotics have many advantages. Basically, prebiotics selectively stimulate the growth of probiotics, which is dose and strain dependent. Prebiotics serve as a selective growth substrate for the probiotics strain during fermentation, during the period of storage, or during its passage through the gut.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

Humectants

A pharmaceutically acceptable humectant can include one or a mixture of humectants, such as, for example, glycerin, sorbitol and polyethylene glycol, for the gummy composition embodiments. The humectant content can be in the range of from about of 1% w/w to about 30% w/w and such as about 2% w/w to about 25% w/w.

Humectants are low molecular weight species that give the sensation of moisture in the gummy composition. The humectants mimic water in the gummy composition and which allows for very low water levels (2-6%) in the gummy composition. The humectant can act as a moisturizing agent in the mouth. The humectant prevents the gummy composition from drying out and helps to maintain softness and shelf life. A very low vapor press is an important property of the humectant for the maintenance of softens so it does not evaporate out. Example humectants are glycerol (glycerin, 1,2,3-propantriol), propylene glycol (1,2-propandiol), aloe vera gel, glyceryl triacetate, and a-hydroxyacids (lactic acid).

In one embodiment, the humectant comprises glycerol. Applicant notes that glycerol extends the shelf life of gummy composition by helping gummies retain their softness and chewability for longer period.

Sweeteners

Example sugar substitutes include saccharin, aspartame, sucralose and acesulfame K. In some embodiments, the sweetener used in the composition not only provides sweetness but also decreases the populations of bacteria in the mouth that lead to oral health problems.

In one embodiment, the sweetener is selected from one or more of saccharin, aspartame, cyclamate, sucralose, Stevia, mannitol, sorbitol, xylitol and similar glycols.

Plasticizers

Plasticizers impart flexibility by lowering the glass transition temperature of the polymers. They act in gummy composition decrease brittleness and increase chewiness. Some plasticizers that can be added to the chewable are lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium and potassium stearates.

Flavoring Agents

A pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, such as spearmint essential oil. Some flavoring agents can also act as sweeteners and can be use as such and include, for example, neohespiridin dehydrochalcone, xylitol, Sucralose, and mixtures thereof, such as xylitol. The flavoring agent content can be in the range of from about of 0.05% w/w to about 3% w/w and such as about 0.5% w/w to about 1% w/w.

Flavors and colors are for sensory appeal. Flavor components in gum exist in liquid, powder or micro-encapsulated forms. Liquid flavor incorporations are either water-soluble, oil-soluble, or water-dispersible emulsions. The oil-soluble flavors remain in the gum longer, resulting in longer lasting flavor sensations, because the gum base is hydrophobic and attracted to oil-based components. Conversely, water soluble flavors dissolve into saliva and as such are extract out of the gum which leads to quicker loss in flavor.

In one embodiment, the flavoring agent is a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

Flavoring Modulating Agents

Taurine, or 2-aminoethanesulfonic acid, is an organic compound that is widely distributed in animal tissues. It is a major constituent of bile and can be found in the large intestine, and accounts for up to of total human body weight. Taurine can reduce bitterness by 50% when added at a concentration of 300 mM.

Coatings

A coating can be applied to the chewable composition. Alternatively, the gummy composition can be wrapped in wax paper. The coating can include sugars such allulose, sorbose, tagatose, trehalose, and isomaltulose or sugar alcohols such as erythritol, sorbitol, mannitol, maltitol, isomalt, and xylitol. Resistant starches may also be used for coating.

Preservatives

A pharmaceutically acceptable preservative can include one or a mixture of preservatives, such as, for example, benzoic acid, sodium benzoate, methylparaben, propylparaben, sorbic acid and potassium sorbate. These preservative may present at levels ranging from about 0.01% w/w to about 2% w/w.

### EXAMPLES

### Example. Tadalafil Low GI Pectin Gummy

78.0 grams pectin; 1020.5 grams Allulose; 538.0 grams Trehalose, 362.0 grams Ismaltulose, 30.0 grams, Mannitol, 3.0 grams Sodium Citrate; 35.0 grams beta-cyclodextrin; 150 grams Maltodextrin, 10.00 grams Tadalafil; Sodium Dodecyl Sulfate, Citric Acid; Malic Acid; CFR Title 21 Granular Pineapple Flavor; CFR Title 21 FD&C Yellow.

In a separate container is added the allulose, trehalose, isomaltulose, maltodextrin, and mannitol. The carbohydrates are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium dodecyl sulfate and sodium citrate. This is mix 2. water is added to a container and heated to 99° C. Mix 2 is added and stirred. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Tadalafil Low GI Pectin Gummy

75.0 grams pectin; 1020.5 grams Allulose; 538.0 grams Trehalose, 362.0 grams Ismaltulose, 30.0 grams, Mannitol, 3.0 grams Sodium Citrate; 35.0 grams beta-cyclodextrin; 200 grams Maltodextrin, 6.0 grams Tadalafil; Sodium Dodecyl Sulfate, Citric Acid; Malic Acid; CFR Title 21 Granular Pineapple Flavor; CFR Title 21 FD&C Yellow.

In a separate container is added the allulose, trehalose, isomaltulose, maltodextrin, and mannitol. The carbohydrates are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium dodecyl sulfate and sodium citrate. This is mix 2. water is added to a container and heated to 99° C. Mix 2 is added and stirred for 2 hours. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Tadalafil Gummy

75.0 grams pectin; 1050.5 grams Maltitol; 840.5 grams Isomalt, 180 grams Maltodextrin, 30.0 grams Mannitol, 3.0 grams Sodium Citrate; 35.0 grams beta-cyclodextrin; 10.00 grams Tadalafil, Sodium Dodecyl Sulfate, Citric Acid; Malic Acid; CFR Title 21 Granular Pineapple Flavor; CFR Title 21 FD&C Yellow Color.

In a separate container is added the maltitol, cyclodextrin, isomalt, maltodextrin and mannitol. The three are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium dodecyl sulfate and sodium citrate. This is mix 2. water is added to a container and heated to 99° C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. Sugar Free Tadalafil Gummy

76.0 grams pectin; 1050.5 grams Maltitol; 840.5 grams Isomalt, 180 grams Maltodextrin, 30.0 grams Mannitol, 3.0 grams Sodium Citrate; 30.0 grams beta-cyclodextrin; 5.00 grams Tadalafil, 0.5 gram Sodium Dodecyl Sulfate, Acid; Malic Acid; 12 grams CFR Title 21 Granular Pineapple Flavor; 5 grams CFR Title 21 FD&C Yellow Color.

In a separate container is added the maltitol, cyclodextrin, isomalt, maltodextrin and mannitol. The three are combined and mixed until homogeneous. This is mix 1. Mix 1 is added to a separate container and water is added. The mixture is heated and begins to boil to create a hot syrup solution. The malic and citric acids are added to the syrup. In a separate container is added the tadalafil, sodium dodecyl sulfate and sodium citrate. This is mix 2. water is added to a container and heated to 99° C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin mixture is then added to the hot syrup. The mixture is allowed to heat until Brix 82 at which time the flavor and color are added. The solution is heated to Brix 83 and then added to molds.

### Example. 10 mg Tadalafil Gummy

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; Orange Flavor USP; 1.36 grams Tadalafil; 20.0 grams 50% Citric Acid solution (in 50% glycerol/water); glycerol; Root Beer flavor; 0.6 grams brown caramel color

In a separate container is added the pectin, 100 grams of sucrose, tadalafil, cyclodextrin and the sodium citrate. The components are mixed until homogeneous. This is mix 1. In a separate container is added the remaining sucrose, fructose, and mannitol. The components are mixed until homogeneous. This is mix 2. In a separate container is added the citric acid solution, brown color, and root beer flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example 2. 10 mg Tadalafil Gummy

40 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 2.80 grams Tadalafil; 20.0 grams 50% Citric Acid solution (in 50% glycerol/water); glycerol; Bourbon Flavor; brown caramel color

In a separate container is added the pectin, 100 grams of sucrose, ½ cyclodextrin and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil hydrochloride, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example. Sugar Free 10 mg Tadalafil Gummy

75.0 grams pectin; 1140.5 grams Maltitol; 930.5 grams Isomalt, 200 grams Maltodextrin, 30.0 grams Mannitol, 3.0 grams Sodium Citrate; 35.0 grams alpha-cyclodextrin; 11.00 grams Tadalafil; Citric Acid; Malic Acid; Pineapple Flavor; Yellow Color.

In a separate container is added the maltodextrin, maltitol, isomalt and mannitol. The three are combined and mixed until homogeneous. This is mix 1. Mix 1 is added to a separate container and water is added. The container temperature is set to 270 F. The mixture is heated and begins to boil to create a hot syrup solution. The malic and citric acids are added to the syrup. In a separate container is added the tadalafil, sodium citrate, and cyclodextrin. This is mix 2. water is added to a container and heated to 99° C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy

75.0 grams pectin; 1140.5 grams Maltitol; 930.5 grams Isomalt, 200 grams Soluble Corn Fiber, 30.0 grams Mannitol, 3.0 grams Sodium Citrate; 35.0 grams cyclodextrin; 11.00 grams Tadalafil; Citric Acid; 4.0 grams Malic Acid; Pineapple Flavor; Yellow Color.

In a separate container is added the soluble corn fiber, maltitol, isomalt and mannitol. The three are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium citrate, and cyclodextrin. This is mix 2. water is added to a container and heated to 99° C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy

Water; 75.0 grams pectin; 1140.5 grams Maltitol; 930.5 grams Isomalt, 200 grams Soluble Tapioca Fiber, grams Mannitol, 3.0 grams Sodium Citrate; 35.0 grams alpha-cyclodextrin; 11.00 grams Tadalafil; 8 grams Citric Acid; 4.0 grams Malic Acid; 12 grams Pineapple Flavor; 6 grams Yellow Color.

In a separate container is added the soluble tapioca fiber, maltitol, isomalt and mannitol. The three are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium citrate, and cyclodextrin. This is mix 2. water is added to a container and heated to 99° C. Mix 2 is added to the water with stirring. This solution is allowed to stir for the 30 minutes. In a separate container is added the pectin. 300 grams from mix 1 is added to the pectin and mixed until homogeneous. This is Mix 3. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy

75.0 grams pectin; 1140.5 grams Maltitol; 930.5 grams Isomalt, 200 grams Inulin, 30.0 grams Mannitol, 3.0 grams Sodium Citrate; 35.0 grams alpha-cyclodextrin; 11.00 grams Tadalafil; Citric Acid; Malic Acid; 2 grams Pineapple Flavor; Yellow Color.

In a separate container is added the inulin, maltitol, isomalt and mannitol. The three are combined and mixed until homogeneous. This is mix 1. Mix 1 is added to a separate container and water is added. The container temperature is set to 270° F. The mixture is heated and begins to boil to create a hot syrup solution. The malic and citric acids are added to the syrup. In a separate container is added the tadalafil, sodium citrate, and cyclodextrin. This is mix 2. water is added to a container and heated to 99° C. Mix 2 is added to the water with stirring. In a separate container is added the pectin. 300 grams from mix 1 is added to the pectin and mixed until homogeneous. This is Mix 3. Mix 3 is added to the water solution of Mix 2 with stirring. Mix 3 is added to the water solution of Mix 2 with stirring. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy

75.0 grams pectin; 1140.5 grams Maltitol; 930.5 grams Isomalt, 30.0 grams Mannitol, 3.0 grams Sodium Citrate; cyclodextrin; 10.00 grams Tadalafil; 4 grams Citric Acid; 4.0 grams Malic Acid; 8 grams Pineapple Flavor; Chili Flavor: Yellow Color.

In a separate container is added the maltitol, isomalt and mannitol. The three are combined and mixed until homogeneous. This is mix 1. In a separate container is added the tadalafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for minutes. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy

75.0 grams pectin; 1140.5 grams Maltitol; 930.5 grams Isomalt, 30.0 grams Mannitol, 3.0 grams Sodium Citrate; 35.0 grams cyclodextrin; 10.00 grams Tadalafil; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the tadalafil, sodium citrate, and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds.

### Example. Sugar Free 5 mg Tadalafil Gummy

75.0 grams pectin; 1140.5 grams Maltitol; 930.5 grams Isomalt, 30.0 grams Mannitol, 3.0 grams Sodium Citrate; 35.0 grams □-cyclodextrin; 5.00 grams Tadalafil; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the tadalafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The pectin container is then added to the hot syrup. The mixture is allowed to heat until Brix 82 at which time the flavor and color are added. The solution is heated to Brix 83 and then added to molds.

### Example. Sugar Free 5 mg Tadalafil Gummy

75.0 grams pectin; 1140.5 grams Maltitol; 930.5 grams Isomalt, 200 grams Maltodextrin, 30.0 grams Mannitol, 3.0 grams Sodium Citrate; 35.0 grams alpha-cyclodextrin; 5.50 grams Tadalafil; 4 grams Citric Acid; 4.0 grams Malic Acid; 12 grams CFR Title 21 Granular Orange Flavor; 6 grams CFR Title 21 beta-Carotene Orange Color.

In a separate container is added the maltodextrin, maltitol, isomalt and mannitol. The three are combined and mixed until homogeneous. This is mix 1. In a separate container is added the tadalafil, sodium citrate, and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds.

### Example. Sugar Free 5 mg Tadalafil Gummy

75.0 grams pectin; 1140.5 grams Maltitol; 930.5 grams Isomalt, 200 grams Inulin, 30.0 grams Mannitol, 3.0 grams Sodium Citrate; 35.0 grams cyclodextrin; 5.50 grams Tadalafil; 4 grams Citric Acid; 4.0 grams Malic Acid; 12 grams CFR Title 21 Granular Orange Flavor; 6 grams CFR Title 21 beta-Carotene Orange Color.

In a separate container is added the inulin, maltitol, isomalt and mannitol. The three are combined and mixed until homogeneous. This is mix 1. In a separate container is added the tadalafil, sodium citrate, and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for minutes. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy with Zinc

75.0 grams pectin; 1140.5 grams Maltitol; 930.5 grams Isomalt, 200 grams Soluble Tapioca Fiber, 30.0 grams Mannitol, 3.0 grams Sodium Citrate; 35.0 grams cyclodextrin; 11.00 grams Tadalafil; 2.10 grams Zinc Sulfate, 8 grams Citric Acid; 4.0 grams Malic Acid; 12 grams Pineapple Flavor; Yellow Color.

In a separate container is added the soluble tapioca fiber, maltitol, isomalt and mannitol. The three are combined and mixed until homogeneous. This is mix 1. In a separate container is added the tadalafil, sodium citrate, zinc sulfate and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The pectin container is then added to the hot syrup. The mixture is allowed to heat until Brix 83 at which time the flavor and color are added. The syrup is then added to molds to yield gummies that were 3200 mg is mass.

### Example. Sugar Free 10 mg Tadalafil Gummy with Zinc

75.0 grams pectin; 1140.5 grams Maltitol; 930.5 grams Isomalt, 200 grams Inulin, 30.0 grams Mannitol, 3.0 grams Sodium Citrate; 35.0 grams beta-cyclodextrin; 11.00 grams Tadalafil; 2.10 grams Zinc Sulfate, 8 grams Citric Acid; 4.0 grams Malic Acid; 12 grams Pineapple Flavor; 6 grams Yellow Color.

In a separate container is added the inulin, maltitol, isomalt and mannitol. The three are combined and mixed until homogeneous. This is mix 1. In a separate container is added the tadalafil, sodium citrate, zinc sulfate and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The pectin container is then added to the hot syrup. The mixture is allowed to heat until Brix 83 at which time the flavor and color are added. The syrup is then added to molds to yield gummies that were 3200 mg is mass.

### Example. 10 mg Tadalafil Gummy with Zinc

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 2.80 grams Tadalafil; 0.400 grams zinc sulfate, 20.0 grams 50% Citric Acid solution (in 50% glycerol/water); 5.0 grams glycerol; 4.5 grams Bourbon Flavor; 0.6 grams brown caramel color

In a separate container is added the pectin, sucrose, ½ cyclodextrin, zinc sulfate and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil hydrochloride, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses. The solution is brought to a boil. Mix 2 is added to the boiling glucose syrup is then added to the boiling pectin/sugars/oil mix. The mixture was heated to Brix 83 at which time Mix 3 was added dropwise with stirring. The molding mixture was then added to silicone molds.

### Example. Sugar Free 10 mg Tadalafil Gummy with Vardenafil

75.0 grams pectin; 1140.5 grams Maltitol; 930.5 grams Isomalt, 200 grams Inulin, 30.0 grams Mannitol, 3.0 grams Sodium Citrate; 35.0 grams alpha-cyclodextrin; 11.00 grams Tadalafil; 1.10 grams Vardenafil Hydrochloride, 8 grams Citric Acid; 4.0 grams Malic Acid; 12 grams Pineapple Flavor; 6 grams Yellow Color.

In a separate container is added the inulin, maltitol, isomalt and mannitol. The three are combined and mixed until homogeneous. This is mix 1. In a separate container is added the tadalafil, vardenafil hydrochloride, sodium citrate, and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example 16. Sugar Free 10 mg Tadalafil Gummy with Vardenafil

75.0 grams pectin; 1140.5 grams Maltitol; 930.5 grams Isomalt, 30.0 grams Mannitol, 3.0 grams Sodium Citrate; 35.0 grams □-cyclodextrin; 11.00 grams Tadalafil; 1.10 grams Vardenafil Hydrochloride, 8 grams Citric Acid; 4.0 grams Malic Acid; 12 grams Pineapple Flavor; 6 grams Yellow Color.

In a separate container is added the maltitol, isomalt and mannitol. The three are combined and mixed until homogeneous. This is mix 1. In a separate container is added the tadalafil, vardenafil hydrochloride, sodium citrate, and cyclodextrin. This is mix 2. In a separate container is added the pectin. 300 grams from mix 1 is added to the pectin and mixed until homogeneous. This is Mix 3. water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. 10 mg Tadalafil Gummy with Vardenafil

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 2.80 grams Tadalafil; 0.200 grams vardenafil hydrochloride, 20.0 grams 50% Citric Acid solution (in 50% glycerol/water); 5.0 grams glycerol; 4.5 grams Bourbon Flavor; 0.6 grams brown caramel color

In a separate container is added the pectin, 100 grams of sucrose, ½ cyclodextrin, zinc sulfate and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil, vardenafil hydrochloride, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 83 at which time Mix 3 was added dropwise with stirring. The molding mixture was then added to silicone molds.

### Example. Sugar Free 10 mg Tadalafil Gummy with Sildenafil

75.0 grams pectin; 1140.5 grams Maltitol; 930.5 grams Isomalt, 200 grams Inulin, 30.0 grams Mannitol, 3.0 grams Sodium Citrate; 35.0 grams O-cyclodextrin; 11.00 grams Tadalafil; 1.10 grams Sildenafil Citrate, 8 grams Citric Acid; 4.0 grams Malic Acid; 12 grams Pineapple Flavor; 6 grams Yellow Color.

In a separate container is added the inulin, maltitol, isomalt and mannitol. The three are combined and mixed until homogeneous. This is mix 1. In a separate container is added the tadalafil, sildenafil citrate, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. 10 mg Tadalafil Gummy with Sildenafil

40.3 grams pectin; 100.0 grams Sucrose; 3.0 grams Sodium Citrate; 130 Grams Sucrose; 100 grams Fructose; 15.0 grams gamma-cyclodextrin; 20.0 grams Mannitol; 410.0 grams Boling Glucose Syrup; 2.80 grams Tadalafil; 0.200 grams sildenafil citrate, 20.0 grams 50% Citric Acid solution (in 50% glycerol/water); 5.0 grams glycerol; 4.5 grams Bourbon Flavor; 0.6 grams brown caramel color.

In a separate container is added the pectin, 100 grams of sucrose, ½ cyclodextrin, zinc sulfate and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil, sildenafil citrate, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 83 at which time Mix 3 was added dropwise with stirring. The molding mixture was then added to silicone molds.

### Example. Sugar Free 10 mg Tadalafil Gummy with Flibanserin

75.0 grams pectin; 1140.5 grams Maltitol; 930.5 grams Isomalt, 200 grams Inulin, 30.0 grams Mannitol, 3.0 grams Sodium Citrate; 35.0 grams alpha-cyclodextrin; 11.00 grams Tadalafil; 1.10 grams Flibanserin, 8 grams Citric Acid; 4.0 grams Malic Acid; 12 grams Pineapple Flavor; 6 grams Yellow Color.

In a separate container is added the inulin, maltitol, isomalt and mannitol. The three are combined and mixed until homogeneous. This is mix 1. In a separate container is added the tadalafil, flibanserin, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. 10 mg Tadalafil Gummy with Flibanserin

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 2.80 grams Tadalafil; 0.200 grams Flibanserin, 20.0 grams 50% Citric Acid solution (in 50% glycerol/water); 5.0 grams glycerol; 4.5 grams Bourbon Flavor; 0.6 grams brown caramel color.

In a separate container is added the pectin, 100 grams of sucrose, ½ cyclodextrin, and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil, flibanserin, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 83 at which time Mix 3 was added dropwise with stirring. The molding mixture was then added to silicone molds.

### Example. Sugar Free 10 mg Tadalafil Gummy with Mirodenafil

75.0 grams pectin; 1140.5 grams Maltitol; 930.5 grams Isomalt, 200 grams Inulin, 30.0 grams Mannitol, 3.0 grams Sodium Citrate; 35.0 grams alpha-cyclodextrin; 11.00 grams Tadalafil; 1.10 grams Mirodenafil, 8 grams Citric Acid; 4.0 grams Malic Acid; 12 grams Pineapple Flavor; 6 grams Yellow Color.

In a separate container is added the inulin, maltitol, isomalt and mannitol. The three are combined and mixed until homogeneous. This is mix 1. In a separate container is added the tadalafil, mirodenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 83 at which time the flavor and color are added. The syrup is then added to molds to yield gummies that were 3200 mg is mass.

### Example. 10 mg Tadalafil Gummy with Mirodenafil

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 2.80 grams Tadalafil; 0.200 grams Mirodenafil, 20.0 grams 50% Citric Acid solution (in 50% glycerol/water); 5.0 grams glycerol; 4.5 grams Bourbon Flavor; 0.6 grams brown caramel color.

In a separate container is added the pectin, 100 grams of sucrose, ½ cyclodextrin, and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil, mirodenafil, brown color, and bourbon flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 83 at which time Mix 3 was added dropwise with stirring. The molding mixture was then added to silicone molds.

### Example. Sugar Free 10 mg Tadalafil Gummy

75.0 grams pectin; 1140.5 grams Maltitol; 930.5 grams Isomalt, 30.0 grams Mannitol, 3.0 grams Sodium Citrate; 35.0 grams alpha-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, cyclodextrin, isomalt and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. Water is added to a container and heated to 99° C. and the sodium dodecyl sulfate added. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy

75.0 grams pectin; 1050.5 grams Maltitol; 840.5 grams Isomalt, 180 grams Maltodextrin, 30.0 grams Mannitol, 3.0 grams Sodium Citrate; 35.0 grams alpha-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; CFR Title 21 beta-Carotene Orange Color.

In a separate container is added the maltitol, cyclodextrin, isomalt, maltodextrin and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. Water is added to a container and heated to 99° C. and the sodium dodecyl sulfate added. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup.

### Example. Sugar Free 10 mg Tadalafil Gummy

75.0 grams pectin; 1120.5 grams Maltitol; 930.5 grams Isomalt, 30.0 grams Mannitol, 20 grams N-Acetylglucosamine, 3.0 grams Sodium Citrate; 35.0 grams alpha-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, N-acetylglucosamine, cyclodextrin, isomalt and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. water is added to a container and heated to 99° C. and the sodium dodecyl sulfate added. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy

78 g pectin; 1030.5 grams Maltitol; 840.5 grams Isomalt, 180 grams Maltodextrin, 30.0 grams Mannitol, grams N-Acetylglucosamine, 3.0 grams Sodium Citrate; 35.0 grams alpha-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, maltodextrin, N-acetylglucosamine, cyclodextrin, isomalt and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. water is added to a container and heated to 99° C. and the sodium dodecyl sulfate added. Mix 2 is added to the water with stirring. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The pectin container is then added to the hot syrup. The mixture is allowed to heat until Brix 82 at which time the flavor and color are added. The solution is heated to Brix 83 and then added to molds. Mix 3 is added to the water solution of Mix 2 with stirring. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy

74 pectin; 1140.5 grams Sorbitol; 930.5 grams Xylitol, 30.0 grams Mannitol, 3.0 grams Sodium Citrate; 35.0 grams alpha-cyclodextrin; 10.00 grams Tadalafil; 4 grams Citric Acid; 4.0 grams Malic Acid; 12 grams CFR Title 21 Granular Orange Flavor; 6 grams CFR Title 21 beta-Carotene Orange Color.

In a separate container is added the sorbitol, xylitol and mannitol. The three are combined and mixed until homogeneous. This is mix 1. In a separate container is added the tadalafil, sodium citrate, and cyclodextrin. This is mix 2. Water is added to a container and heated to 99° C. Mix 2 is added to the water with stirring. In a separate container is added the pectin. 300 grams from mix 1 is added to the pectin and mixed until homogeneous. This is Mix 3. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy

72 g pectin; 1120.5 grams Sorbitol; 930.5 grams Xylitol, 30.0 grams Mannitol, 20 grams N-Acetylglucosamine, 3.0 grams Sodium Citrate; 35.0 grams □-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, N-acetylglucosamine, cyclodextrin, isomalt and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. Water is added to a container and heated to 99° C. and the sodium dodecyl sulfate added. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds. Mix 3 is added to the water solution of Mix 2 with stirring. The pectin container is then added to the hot syrup. The mixture is allowed to heat until Brix 82 at which time the flavor and color are added. The solution is heated to Brix 83 and then added to molds.

### Example. Sugar Free 10 mg Tadalafil Gummy

75.0 grams pectin; 1140.5 grams Sorbitol; 930.5 grams Xylitol, 30.0 grams Mannitol, 3.0 grams Sodium Citrate; 35.0 grams alpha-cyclodextrin; 10.00 grams Tadalafil; 1.0 gram Sodium dodecyl sulfate, 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, N-acetylglucosamine, cyclodextrin, isomalt and mannitol. The three are combined and mixed until homogeneous. This is mix 1. In a separate container is added the tadalafil and sodium citrate. This is mix 2. In a separate container is added the pectin. 300 grams from mix 1 is added to the pectin and mixed until homogeneous. This is Mix 3. Water is added to a container and heated to 99° C. and the sodium dodecyl sulfate added. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is heated to Brix 83 and then added to molds.

### Example. 10 mg Tadalafil Gummy

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Glucose Syrup; 2.8 grams Tadalafil; 0.3 grams Sodium dodecyl sulfate, 10 Grams Citric Acid; 4.5 grams Bourbon Flavor; 0.6 grams brown caramel color

In a separate container is added the pectin, 100 grams of sucrose, ½ cyclodextrin and the sodium citrate. The components are mixed until homogeneous. This is mix 1. In a separate container is added the remaining sucrose, ¼ cyclodextrin, fructose, and mannitol. The components are mixed until homogeneous. This is mix 2. In a separate container is added the citric acid solution, ¼ cyclodextrin, tadalafil hydrochloride, brown color, and bourbon flavor. All is mixed and warmed to 175° F. until all is dissolved. This is Mix 3. Water is heated to 200° F. in a saucepan and sodium dodecyl sulfate is added. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 83 at which time Mix 3 was added dropwise with stirring. The gummy syrup was then added to silicone molds.

### Example. 10 mg Tadalafil Gummy

40.3 grams pectin; 100.0 grams Sucrose; 3.0 grams Sodium Citrate; 120 Grams Sucrose; 10 grams N-acetylglucosamine, 100 grams Fructose; 15.0 grams beta-cyclodextrin; 20.0 grams Mannitol; 410.0 grams Glucose Syrup; 2.80 grams Tadalafil; 10 Grams Citric Acid; 4.5 grams Root Beer Flavor; 0.6 grams brown caramel color. [what is N-acetylglucosamine for/]

In a separate container is added the pectin, 100 grams of sucrose, ½ cyclodextrin and the sodium citrate. The components are mixed until homogeneous. This is mix 1. In a separate container is added the remaining sucrose, ¼ cyclodextrin, fructose, N-acetylglucosamine, and mannitol. The components are mixed until homogeneous. This is mix 2. Mix 2 is added to the boiling glucose syrup is then added to the boiling pectin/sugars/oil mix. In a separate container is added the citric acid solution, ¼ cyclodextrin, tadalafil hydrochloride, brown color, and bourbon flavor. All is mixed and warmed to 175° F. until all is dissolved. This is Mix 3. Water is heated to 200° F. in a saucepan and sodium dodecyl sulfate is added. To the hot water is added Mix 1. The mixture is stirred until the pectin fully swells and disperses which takes roughly 3-5 minutes. The solution is brought to a boil. The mixture was heated to Brix 83 at which time Mix 3 was added dropwise with stirring. The gummy syrup was then added to silicone molds.

### Example. 10 mg Tadalafil Gummy

40.3 grams pectin; 100.0 grams Sucrose; 3.0 grams Sodium Citrate; 120 Grams Sucrose; 10 grams N-acetylglucosamine, 100 grams Fructose; 0.3 grams Sodium dodecyl sulfate, 15.0 grams alpha-cyclodextrin; 20.0 grams Mannitol; 410.0 grams Glucose Syrup; 2.80 grams Tadalafil; 10 Grams Citric Acid; 4.5 grams Root Beer Flavor; 0.6 grams brown caramel color

In a separate container is added the pectin, sucrose, ½ cyclodextrin and the sodium citrate. The components are mixed until homogeneous. This is mix 1. In a separate container is added the remaining sucrose, ¼ cyclodextrin, fructose, N-acetylglucosamine, and mannitol. The components are mixed until homogeneous. This is mix 2. Mix 2 is added to the boiling glucose syrup is then added to the boiling pectin/sugars/oil mix. In a separate container is added the citric acid solution, ¼ cyclodextrin, tadalafil hydrochloride, brown color, and bourbon flavor. All is mixed and warmed to 175° F. until all is dissolved. This is Mix 3. Water is heated to 200° F. in a saucepan and sodium dodecyl sulfate is added. To the hot water is added Mix 1. The mixture is stirred until the pectin fully swells. The solution is brought to a boil. The mixture was heated to Brix 83 at which time Mix 3 was added dropwise with stirring. The gummy syrup was then added to silicone molds.

### Example. Sugar Free 30 mg Sildenafil Gummy

75.0 grams pectin; 1140.5 grams Maltitol; 930.5 grams Isomalt, 30.0 grams Mannitol, 3.0 grams Sodium Citrate; 50.0 grams □-cyclodextrin; 31.50 grams Sildenafil Citrate; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the sildenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. Sugar Free 25 mg Mirodenafil Gummy

75.0 grams pectin; 1140.5 grams Maltitol; 930.5 grams Isomalt, 30.0 grams Mannitol, 3.0 grams Sodium Citrate; 50.0 grams □-cyclodextrin; 25.4 grams Mirodenafil; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the mirodenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. Sugar Free 25 mg Fibanserin Gummy

75.0 grams pectin; 1140.5 grams Maltitol; 930.5 grams Isomalt, 30.0 grams Mannitol, 3.0 grams Sodium Citrate; 50.0 grams □-cyclodextrin; 25.4 grams Flibanserin; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the flibanserin, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. 2 mg Vardenafil Gummy

40.3 grams pectin; 100.0 grams Sucrose; 3.0 grams Sodium Citrate; 130 Grams Sucrose; 100 grams Fructose; 15.0 grams alpha-cyclodextrin; 20.0 grams Mannitol; 410.0 grams Boling Glucose Syrup; 1.51 grams vardenafil hydrochloride; 20.0 grams 50% Citric Acid solution (in 50% glycerol/water); 5.0 grams glycerol; 4.5 grams Root Beer flavor; 0.6 grams brown caramel color

In a separate container is added the pectin, 100 grams of sucrose, vardenafil hydrochloride, cyclodextrin and the sodium citrate. The components are mixed until homogeneous. This is mix 1. In a separate container is added the remaining sucrose, fructose, and mannitol. The components are mixed until homogeneous. This is mix 2. In a separate container is added the citric acid solution, brown color, and root beer flavor. All is mixed and warmed to 175° F. until all is dissolved. This is Mix 3. Water is heated to 200° F. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example. Sugar Free 20 mg Vardenafil Gummy

75.0 grams pectin; 1140.5 grams Maltitol; 930.5 grams Isomalt, 30.0 grams Mannitol, 3.0 grams Sodium Citrate; 35.0 grams gamma-cyclodextrin; 20.20 grams Vardenafil Hydrochloride; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the vardenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. Sugar Free 10 mg Vardenafil Gummy

75.0 grams pectin; 1140.5 grams Maltitol; 930.5 grams Isomalt, 30.0 grams Mannitol, 3.0 grams Sodium Citrate; 35.0 grams □-cyclodextrin; 10.0 grams Vardenafil Hydrochloride; 4 grams Citric Acid; 4.0 grams Malic Acid; 12 grams CFR Title 21 Granular Raspberry Flavor; 6 grams CFR Title 21 Watermelon Red Color.

In a separate container is added the maltitol, isomalt and mannitol. The three are combined and mixed until homogeneous. This is mix 1. In a separate container is added the vardenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and 400 grams of water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. 8 mg Sildenafil Gummy

40.3 grams pectin; 100.0 grams Sucrose; 3.0 grams Sodium Citrate; 130 Grams Sucrose; 100 grams Fructose; 15.0 grams □-cyclodextrin; 20.0 grams Mannitol; 410.0 grams Boling Glucose Syrup; 7.65 grams sildenafil citrate; 20.0 grams 50% Citric Acid solution (in 50% glycerol/water); 5.0 grams glycerol; 4.5 grams Raspberry flavor; 0.6 grams Red color

In a separate container is added the pectin, 100 grams of sucrose, sildenafil citrate, cyclodextrin and the sodium citrate. The components are mixed until homogeneous. This is mix 1. In a separate container is added the remaining sucrose, fructose, and mannitol. The components are mixed until homogeneous. This is mix 2. In a separate container is added the citric acid solution, red color, and raspberry flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example. 8 mg Sildenafil Chocolate Gummy

285.0 grams Water; 40.3 grams pectin; 100.0 grams Sucrose; 3.0 grams Sodium Citrate; 130 Grams Sucrose; 100 grams Fructose; 30 grams Cacao Powder, 15.0 grams □-cyclodextrin; 20.0 grams Mannitol; 410.0 grams Boling Glucose Syrup; 8.10 grams sildenafil citrate; 20.0 grams 50% Citric Acid solution (in 50% glycerol/water); 5.0 grams glycerol; 1.2 grams Raspberry flavor

In a separate container is added the pectin, 100 grams of sucrose, sildenafil citrate, cyclodextrin and the sodium citrate. The components are mixed until homogeneous. This is mix 1. In a separate container is added the remaining sucrose, cacao powder, fructose, and mannitol. The components are mixed until homogeneous. This is mix 2. In a separate container is added the citric acid solution, and raspberry flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example. 30 mg Sildenafil Gummy

40.3 grams pectin; 100.0 grams Sucrose; 3.0 grams Sodium Citrate; 130 Grams Sucrose; 100 grams Fructose; 30.0 grams □-cyclodextrin; 20.0 grams Mannitol; 410.0 grams Boling Glucose Syrup; 31.5 grams sildenafil citrate; 20.0 grams 50% Citric Acid solution (in 50% glycerol/water); 5.0 grams glycerol; 4.5 grams Raspberry flavor; 0.6 grams Red color

In a separate container is added the pectin, 100 grams of sucrose, sildenafil citrate, cyclodextrin and the sodium citrate. The components are mixed until homogeneous. This is mix 1. In a separate container is added the remaining sucrose, fructose, and mannitol. The components are mixed until homogeneous. This is mix 2. In a separate container is added the citric acid solution, red color, and raspberry flavor. All is mixed and warmed to 175° F. until all is dissolved. This is Mix 3. Water is heated to 200° F. in a saucepan. To the hot water is added Mix 1. The mixture is stirred until the pectin fully swells and disperses which takes roughly 3-5 minutes. The solution is brought to a boil. Mix 2 is added to the boiling glucose syrup is then added to the boiling pectin/sugars/oil mix. The mixture was heated to Brix 79 at which time Mix 3 was added dropwise with stirring. The molding mixture was then added to silicone molds.

### Example. Sugar Free 10 mg Sildenafil Gummy

75.0 grams pectin; 1140.5 grams Maltitol; 930.5 grams Isomalt, 30.0 grams Mannitol, 3.0 grams Sodium Citrate; 35.0 grams beta-cyclodextrin; 10.0 grams Sildenafil Citrate; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the sildenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

### Example. Sugar Free 20 mg Sildenafil Gummy

75.0 grams pectin; 1140.5 grams Maltitol; 930.5 grams Isomalt, 30.0 grams Mannitol, 3.0 grams Sodium Citrate; 35.0 grams alpha-cyclodextrin; 20.20 grams Sildenafil Citrate; 4 grams Citric Acid; 4.0 grams Malic Acid; 12 grams CFR Title 21 Granular Orange Flavor; 6 grams CFR Title 21 beta-Carotene Orange Color.

In a separate container is added the maltitol, isomalt and mannitol. The three are combined and mixed until homogeneous. This is mix 1. In a separate container is added the sildenafil, sodium citrate, and cyclodextrin. This is mix 2. In a separate container is added the pectin. 300 grams from mix 1 is added to the pectin and mixed until homogeneous. This is Mix 3. Water is added to a container and heated to 99° C. Mix 2 is added to the water with stirring. This solution is allowed to stir for the 30 minutes. Mix 1 is added to a separate container and water is added. The container temperature is set to 270° F. The mixture is heated and begins to boil to create a hot syrup solution. The malic and citric acids are added to the syrup. Mix 3 is added to the water solution of Mix 2 with stirring. The solution is allowed to stir for 5-10 minutes. The pectin container is then added to the hot syrup. The mixture is allowed to heat until Brix 82 at which time the flavor and color are added. The solution is then heated to Brix 83 and then added to molds.

What is claimed is:

1. A chewable composition, comprising,
an active pharmaceutical ingredient (API) composition, comprising a PED5 inhibitor,
a surfactant composition, wherein the surfactant composition and the API composition have a weight ration from about 0.1 to about 15,
a binding composition, comprising a mono- or di-saccharide, a sugar alcohol, an oligosaccharide, or a combination thereof, and
a gelling composition in a sufficient amount to provide a cohesive gelled product.

2. The chewable composition of claim 1, wherein the API composition comprises tadalafil, sildenafil, vardenafil, avanafil, or a combination thereof.

3. The chewable composition of claim 1, wherein the API composition comprises tadalafil from about 2 mg to about 20 mg per dose.

4. The chewable composition of claim 1, wherein the surfactant composition comprises tween 80, sodium dodecyl sulfate, alpha Tocopherol, benzalkonium chloride, benzyl alcohol, cetostearyl Alcohol, cetrimide, cetylpyridinium chloride, docusate dodium, glyceryl monostearate, glyceryl palmitostearate, linoleic acid, macrogol 15 hydroxystearate, myristic acid, myristyl alcohol, macrogol cetostearyl ether, macrogol lauryl ether, macrogol oleyl ether, macrogol stearyl ether, polyoxyethylene alkyl ethers, polyoxyethylene castor oil derivatives, polyoxyethylene sorbitan fatty acid esters, polysorbate 20, polysorbate 40, polysorbate 60, polysorbate 80, polyoxyethylene stearates, polyoxyl 40 stearate, caprylocaproyl macrogolglycerides, lauroyl macrogolglycerides, linoleoyl macrogolglycerides, oleoyl macrogolglycerides, stearoyl macrogolglycerides, polyoxylglycerides, sodium stearyl fumarate, sorbitan esters (sorbitan fatty acid esters), sorbitan laurate, sorbitan oleate, sorbitan palmitate, sorbitan stearate, sorbitan trioleate, sorbitan sesquioleate, stearic acid, stearyl alcohol, or a combination thereof.

5. The chewable composition of claim 1, comprising from 0.1% to 10% by weight of the surfactant composition.

6. The chewable composition of claim 1, wherein the ratio of the API composition and the surfactant composition is from 8 to about 12.

7. The chewable composition of claim 1, further comprising a complexing agent, wherein the complexing agent comprises cyclodextrin, a nucleotide, resistant starch, an amide, a peptide, or a combination thereof.

8. The chewable composition of claim 1, wherein the binding composition comprises glucose, sucrose, fructose, or a combination thereof.

9. The chewable composition of claim 1, further comprising a polymer stabilizer, and wherein the polymer stabilizer comprises a polymer of glucose monomer or mannose monomer linked through glycosidic bonds, wherein the glycosidic bond is substantially free of 1,4-alpha-glycosidic bond, and wherein the binding composition is essentially free of glucose, sucrose, or fructose, wherein the chewable composition.

10. The chewable composition of claim 10, wherein the binding composition comprises sugar alcohol.

11. The chewable composition of claim 10, wherein the binding composition comprises allulose, isomaltose, trehalose, tagatose, raffinose, or a combination thereof.

12. The chewable composition of claim 1, wherein the gelling composition comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

13. The chewable composition of claim 1, wherein the gelling composition comprises pectin having a methoxyl content of less than 15% by weight of pectin.

14. The chewable composition of claim 1, wherein the gelling composition comprises pectin having an amide content of more than 20% by weight of pectin.

15. The chewable composition of claim 1, wherein the gelling composition comprises pectin, gelatin, collagen, or a combination thereof.

16. The chewable composition of claim 1, wherein the gelling composition comprises pectin and collagen in a ratio from about 1:1 to about 1:3.

17. The chewable composition of claim 1, further comprising an herb composition, an antioxidant composition, a vitamin composition, a mineral composition, an amino acid composition, a probiotics composition, or a prebiotics composition.

18. The chewable composition of claim 19, further comprising an additive selected from sweeteners, food acids, flavoring agents, coloring agents, humectants, bulking agents, fatty acids, triglycerides, plasticizers, thickeners, preservatives, or and a mixture thereof.

19. The chewable composition of claim 1, wherein the gelling composition comprises pectin, wherein the pH of the composition is more than 4.

20. A method of making the chewable composition of claim 1, comprising,
dividing the binding composition into a first binding portion and a second binding portion,
combining a first mixture and water and heating to at a first elevated temperature to provide a first solution, wherein the first mixture comprises the first binding portion,
combining the second mixture and water at a second elevated temperature to provide a second solution, wherein the second mixture comprises the gelling composition, the second binding portion, and the complexing agent, wherein the second binding portion is at least twice the mass of the gelling composition,
mixing the second solution into the first solution at a third elevated temperature to provide a third mixture, wherein the third mixture has a Brix number from about 78 to about 86, and
adjusting pH of the third solution with a buffer salt to from about 3 to about 7.

21. The method of claim 20, wherein the first mixture further comprises a polymer stabilizer.

22. The method of claim 20, wherein the second mixture or the third mixture further comprises the API composition, the surfactant composition or both.

23. The method of claim 20, further comprising the step of adding a coloring agent, a flavoring agent, or a combination thereof into the third mixture to provide a molding mix having a Brix from about 78 to about 86.

24. The method of claim 20, further comprising adding the API composition and the surfactant composition to the molding mixture.

25. A method for treating erectile dysfunction in a subject, benign prostatic hyperplasia in a subject, or pulmonary arterial hypertension in a subject or comprising administrating an effective amount of the chewable composition of claim 1 to the subject.
